(19) United States
(12) Patent Application Publication
(10) Pub. No.: US 2010/0280024 A1
(43) Pub. Date: Nov. 4, 2010

(54) RENIN INHIBITORS

(76) Inventors: Jigar Desai, Gurarat (IN); Pravin S. Thombare, Gurarat (IN); Mukul R. Jain, Gurarat (IN); Pankaj Ramanbhai Patel, Gurarat (IN)

Correspondence Address: LADAS & PARRY LLP, 26 WEST 61ST STREET, NEW YORK, NY, 10023, US

(21) Appl. No.: 12/682,720

(22) PCT Filed: Oct. 13, 2008
(86) PCT No.: PCT/IN08/00662

§ 371 Date: Jul. 9, 2010

(30) Foreign Application Priority Data
Oct. 15, 2007 (IN) 2045/MUM/2007
Apr. 7, 2008 (IN) 803/MUM/2008

(51) Int. Cl. A61K 31/536; C07D 215/14; A61K 31/47; C07D 217/00; C07D 265/36; C07D 211/32; A61K 31/445; C07D 403/02; A61K 31/506; C07D 241/04; A61K 31/495; C07D 207/00; A61K 31/40; C07D 295/00; A61K 31/5375; A61K 31/4709; C07D 401/00; A61K 31/4545; A61P 9/12

(52) U.S. Cl. 514/230.5; 546/174; 514/311; 546/146; 514/307; 544/105; 546/235; 514/331; 544/295; 514/252.14; 544/399; 514/252.12; 548/566; 514/428; 544/162; 514/231.2; 514/314; 546/194; 514/318

(57) ABSTRACT

The present invention relates to renin inhibitors of formula (I), their pharmaceutically acceptable salts and pharmaceutical compositions containing them. The present invention also relates to a process of preparing compounds of general formula (I), their tautomeric forms, their pharmaceutically acceptable salts, pharmaceutical compositions containing them, and novel intermediates involved in their synthesis.

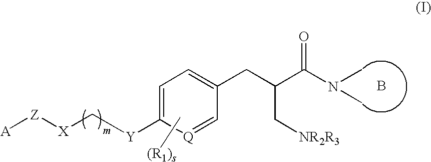

### FIELD OF INVENTION

The present invention relates to novel renin inhibitors of general formula (1), novel intermediates involved in their synthesis, their pharmaceutically acceptable salts and pharmaceutical compositions containing them. The present invention also relates to a process of preparing compounds of general formula (1), their tautomeric forms, their pharmaceutically acceptable salts, pharmaceutical compositions containing them, and novel intermediates involved in their synthesis.

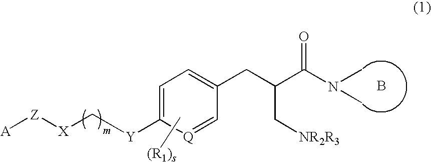

### BACKGROUND TO THE INVENTION

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT1 and AT2. Whereas AT1 seems to transmit most of the known functions of Ang II, the role of AT2 is still unknown.

Modulation of the RAS represents a major advance in the treatment of cardiovascular diseases. ACE inhibitors and AT1 blockers have been accepted to treat hypertension (Waeber B. et al, “The renin-angiotensin system: role in experimental and human hypertension”, in Birkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al, Kidney International, 1994, 45, 403; Breyer J. A. et al. Kidney International, 1994, 45, S156), in the prevention of congestive heart failure (Vaughan D. E. et al, Cardiovasc. Res., 1994, 28, 159; Fouad-Tarazi F. et al., Am. J. Med, 1988, 84 (Suppl. 3A), 83) and myocardial infarction (Pfeffer M. A. et al, N. Engl. J. Med., 1992, 327, 669).

Interest in the development of renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1 155). In patients inhibition of ACE thus leads to bradykinin accumulation causing cough (5-20%) and potentially life-threatening angioneurotic edema (0.1-0.2%) (Israili Z. H. et al, Annals of Internal Medicine, 1992, 117, 234). Chymase is not inhibited by ACE inhibitors. Therefore, the formation of Ang II is still possible in patients treated with ACE inhibitors. Blockade of the AT1 receptor (e.g. by losartan) on the other hand overexposes other AT-receptor subtypes (e.g. AT₂) to Ang II, whose concentration is significantly increased by the blockade of AT1 receptors. In summary, renin inhibitors are expected to demonstrate a different pharmaceutical profile than ACE inhibitors and AT1 blockers with regard to efficacy in blocking the RAS and in safety aspects. Only limited clinical experience (Azizi M. et al., J. Hypertens., 1994, 12, 419; Neutel J. M. et al, Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. One compound containing four chiral centers has entered clinical trials (Rahuel J. et al. Chem. Biol., 2000, 7, 493; Mealy N. E., Drugs of the Future, 2001, 26, 1139). Thus, renin inhibitors with good oral bioavailability and long duration of action are required. The first non-peptide renin inhibitors were described which show high in vitro activity (Oefner C. et al, Chem. Biol, 1999, 6, 127; Patent Application WO97/09311; Marki H. P. et al., 11 Farmaco, 2001, 56, 21). Recently amine based non-peptidic renin inhibitors have been disclosed in WO 2007099509, having the following general structure

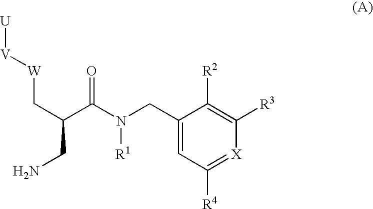

wherein R¹ represents C₁₋₇ alkyl or cycloalkyl group preferably cycloalkyl such as cyclopropyl groups and in WO 2007009250 having the following general formula

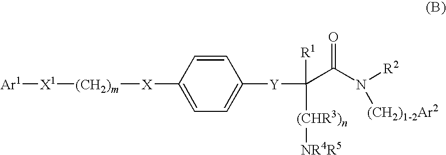

wherein R² is selected from group H, C₁₋₆ alkyl, C₂₋₆ alkenyl, C₁₋₆ alkoxy, CF₃, CH₂CF₃ groups. Compounds disclosed in both the applications were reported to show potency in nanomolar range.

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis. So, the present invention describes these non-peptidic renin inhibitors

### SUMMARY OF THE INVENTION

The present invention describes a group of novel compounds as renin inhibitors useful for the treatment cardiovascular events, renal insufficiency and other related diseases. The novel compounds are defined by the general formula (1) below:

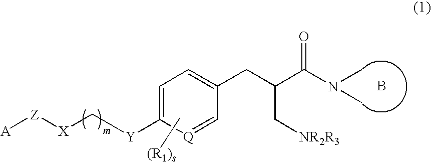

The compounds of the present invention are useful in the treatment of the human or animal body, by regulating renin levels. The compounds of this invention are therefore suitable for the treatment of cardiovascular events, renal insufficiency other related diseases

### EMBODIMENTS OF THE PRESENT INVENTION

The main objective of the present invention thus is to provide novel compounds of general formula (1), novel intermediates involved in their synthesis, their pharmaceutically acceptable salts, and pharmaceutical compositions containing them or their mixtures as therapeutic agents.

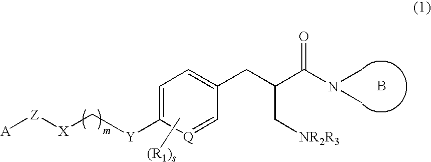

In an embodiment is provided processes for the preparation of novel compounds of general formula (1), novel intermediates involved in their synthesis, their pharmaceutically acceptable salts, and pharmaceutical compositions containing them.

In another embodiment is provided pharmaceutical compositions containing compounds of general formula (1), their pharmaceutically acceptable salts, comprising pharmaceutically acceptable carriers, solvents, diluents, excipients and other media normally employed in their manufacture.

In a further embodiment is provided the use of the novel compounds of the present invention as blood pressure regulating agents, by administering a therapeutically effective & non-toxic amount of the compounds of formula (1) or their pharmaceutically acceptable compositions to the mammals.

### DETAILED DESCRIPTION

The present invention therefore discloses renin inhibitors of formula (I) below

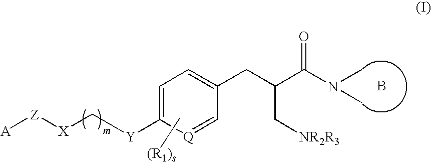

wherein m is an integer selected from 1 to 4; Z represents either a bond or —CH₂—; X and Y are each independently selected from the group comprising of —CH₂—, O, and S(O)ₚ; p in each instance in which it occur is independently 0, 1, 2;
‘A’ is an optionally substituted aryl or a 5-6 membered heterocyclic ring containing 1 to 3 heteroatom selected from O, S, and N, wherein ‘A’ may be substituted with one, two, three or four substituents independently selected from the group comprising of OH, CN, halogen, N₃, NO₂, COOH, OCF₂H, CF₃, C₁-C₆alkyl, C₂-C₆alkenyl, C₁-C₆alkoxy, C(O)C₁-C₆ alkyl and S(O)ₚ C₁-C₆ alkyl, (CH₂)₁₋₂Oalkyl groups;
Preferred group representing ‘A’ may be selected from phenyl, benzyl, piperidinyl groups;
Q represents carbon or nitrogen atoms;
‘B’ represents a saturated, unsaturated or partly unsaturated single or fused heterocyclic ring which may optionally contain one or more additional heteroatoms selected from nitrogen, oxygen or sulphur or may comprise an —SO— or an —SO₂-group. When the heterocyclic ring as defined above comprises one or more nitrogen atom, such nitrogen atom may optionally be substituted with optionally substituted groups selected from C₁-C₈-alkyl, C ₁-C₈-alkanoyl, aryl or heterocyclic group. When ‘B’ represents a fused ring then the ring system preferably contains 9-16 member heterocyclic groups. The heterocycle ‘B’, as defined above, may optionally be substituted with groups selected from halogen, hydroxyl, oxide, oxo, cyano, optionally substituted groups selected from haloalkyl, haloalkoxy, C₁-C₈-alkyl, C₁-C₈-alkoxy, C₁-C₈-alkoxy-C₁-C₈-alkyl, C₁-C₈-alkoxy-C₁-C₈-alkoxy, C₁-C₈-alkoxycarbonylamino, C₁-C₈-alkylcarbonylamino, C₁-C₈-alkylamino, N,N-di-C₁-C₈-alkylamino, aryl-C₀-C₄-alkyl, aryloxy-C₀-C₄-alkyl, aryl-C₀-C₄-alkyl-C₁-C₈-alkoxy, aryloxy-C₀-C₄-alkyl-C₁-C₈-alkoxy, heterocyclyl-C₀-C₄-alkyl, heterocyclyloxy-C₀-C₄-alkyl, heterocyclyl-C₀-C₄-alkyl-C₁-C₈-alkoxy or heterocyclyloxy-C₀-C₄-alkyl-C₁-C₈-alkoxy groups;

Preferred groups representing ‘B’ selected to form an optionally substituted 1,2,3,4-tetrahydroquinoline, 1,2,3,4-tetrahydroisoquinoline, piperidine, morpholine, pyrrolidine, piperazine, indoline and indole & their suitable derivatives; R₁ at each occurrence independently represents hydrogen, halogen, cyano, optionally substituted C₁-C₆ alkyl, C₁-C₆ alkoxy groups; ‘s’ represents integers from 0, 1, 2 and 3;

R₂, R₃ may be same or different and independently selected from the group comprising of hydrogen, optionally substituted C₁-C₆ alkyl, C₁-C₆ alkoxy, C(O)R_{b}, C(O)NR_{c}R_{d} groups; R_{b} is selected from the group comprising of hydrogen, optionally substituted C₁-C₆ alkyl, C₁-C₆ alkoxy groups;

Rₑ and R_{d} are independently selected from the groups comprising hydrogen, optionally substituted C₁-C₆ alkyl, unsubstituted or substituted aryl groups;

As used herein, the term “heterocycle” or “heterocyclic system” is intended to mean a stable 5- to 8-membered monocyclic or bicyclic or a 9- to 16-membered polycyclic heterocyclic ring which may be saturated, partially saturated or unsaturated, and which consists of carbon atoms and further comprises from 1 to 4 heteroatoms independently selected from the group consisting of N, O and S and including any bicyclic group in which any of the above-defined heterocyclic rings is fused to a benzene ring. The term heterocycle is intended to include both aromatic as well as non-aromatic ring system. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic system” is intended to mean a stable 5- to 7-membered monocyclic or bicyclic or 9- to 16-membered polycyclic heterocyclic aromatic ring which consists of carbon atoms and from 1 to 4 heteroatoms independently selected from the group consisting of N, O and S. It is preferred that the total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and Spiro compounds containing, for example, the above heterocycles.

Suitable substituents wherever applicable includes, but are not limited to the following radicals, alone or in combination with other radicals, hydroxyl, oxo, halo, thio, nitro, amino, alkyl, alkoxy, haloalkyl or haloalkoxy groups;

The term “substituted,” as used herein, means that any one or more hydrogens on the designated atom is replaced with a selection from the indicated group, provided that the designated atom's normal valency is not exceeded, and that the substitution results in a stable compound.

In a further embodiment the groups, radicals described above may be selected from:
the “alkyl” group used either alone or in combination with other radicals, denotes a linear or branched radical containing one to six carbons, selected from methyl, ethyl, n-propyl, iso-propyl, n-butyl, sec-butyl, tert-butyl, amyl, t-amyl, n-pentyl, n-hexyl, and the like;
The term “alkenyl” used herein, either alone or in combination with other radicals, denotes a linear or branched radical containing two to twelve carbons; such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like. The term “alkenyl” includes dienes and trienes of straight and branched chains;
The term “alkoxy” used herein, either alone or in combination with other radicals, denotes a radical alkyl, as defined above, attached directly to an oxygen atom, such as methoxy, ethoxy, n-propoxy, iso-propoxy, n-butoxy, t-butoxy, iso-butoxy, pentyloxy, hexyloxy, and the like;
The term “halo” or “halogen” used herein, either alone or in combination with other radicals, such as “haloalkyl”, “perhaloalkyl” etc, refers to a fluoro, chloro, bromo or iodo group. The term “haloalkyl” denotes an alkyl radical, as defined above, substituted with one or more halogens; such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy, chloromethoxy, fluoroethoxy chloroethoxy groups, and the like.
The term “aryl” refers to aromatic monocyclic ring system. Suitable aryl group include phenyl, naphthyl and the like.

The term “C₀” as employed in expressions such as “C₀-C₄ alkyl” means a direct covalent bond. Similarly, when an integer defining the presence of certain number of atoms in a group is equal to zero, it means that the atom adjacent thereto is connected directly by a bond.

The compound of the present invention may have chiral centers, e.g. one chiral center [providing for two stereoisomer, (R) and (S)], or two chiral centers [providing up to four stereoisomers (R,R), (S,S), (R,S), (S,R)]. This invention includes all the optical isomers and mixture thereof. Unless specifically mentioned otherwise, reference to one isomer applies to any of the possible isomers. Whenever the isomeric composition is unspecified, all the possible isomers are included.

The present invention also relates to pro-drugs of a compound of formula (1) that convert in vivo to the compound of formula (1) as such. Any reference to a compound of formula (1) is therefore to be understood as referring also to the corresponding pro-drugs of the compound of formula (1), as appropriate and expedient.

### List of Abbreviation

Boc: t-butyloxycarbonyl
DMF: Dimethyl formamide
DMSO: Dimethyl sulfoxide
THF: Tetrahydrofuran
DCM: Dichloromethane
EDAC.HCl: N-(3-Dimethyl aminopropyl)-N′-ethyl carbodiimide hydrochloride,
HOBT: 1-Hydroxy benzotriazole
TFA: Trifluoro acetic acid
DCC: Dicyclohexylcarbodiimide
DIEA: Disopropyl ethyl amine
EtOAc: Ethyl acetate
h: Hour(s)
min: minute(s)
t_{Ret}: Retention time

Suitable groups and substituents on the groups may be selected from those described anywhere in the specification.

Particularly useful compounds may be selected from
3-Amino-1-(3,4-dihydro-2H-quinolin-1-yl)-2-{4-[2-(2,5-dimethyl-phenoxy)-ethoxy]-benzyl}-propan-1-one;
3-Amino-2-{4-[2-(2,6-dichloro-4-methyl-phenoxy)-ethoxy]-benzyl}-1-(3,4-dihydro-2H-quinolin-1-yl)-propan-1-one;
3-Amino-2-{4-[2-(2,6-dichloro-4-methyl-phenoxy)-ethoxy]-benzyl}-1-(3,4-dihydro-1H-isoquinolin-2-yl)-propan-1-one;
3-Amino-2-{4-[2-(2,6-dichloro-4-methyl-phenoxy)-ethoxy]-benzyl}-1-(2,3-dihydro-benzo[1,4]oxazin-4-yl)-propan-1-one;
3-Amino-1-(6-chloro-3,4-dihydro-2H-quinolin-1-yl)-2-{4-[2-(2,6-dichloro-4-methyl-phenoxy)-ethoxy]-benzyl}-propan-1-one;
3-Amino-1-(6-bromo-3,4-dihydro-2H-quinolin-1-yl)-2-{4-[2-(2,6-dichloro-4-methyl-phenoxy)-ethoxy]-benzyl}-propan-1-one;
3-Amino-1-(7,8-dichloro-3,4-dihydro-2H-quinolin-1-yl)-2-{4-[2-(2,6-dichloro-4-methyl-phenoxy)-ethoxy]-benzyl}-propan-1-one;
3-Amino-2-{4-[2-(2,6-dichloro-4-methyl-phenoxy)-ethoxy]-benzyl}-1-(8-methoxy-3,4-dihydro-2H-quinolin-1-yl)-propan-1-one;
3-Amino-2-{4-[2-(2,6-dichloro-4-methyl-phenoxy)-ethoxy]-benzyl}-1-(7,8-dimethyl-3,4-dihydro-2H-quinolin-1-yl)-propan-1-one;
3-Amino-2-{4-[2-(2,6-dichloro-4-methyl-phenoxy)-ethoxy]-benzyl}-1-(6-methyl-3,4-dihydro-2H-quinolin-1-yl)-propan-1-one;
3-Amino-2-{4-[2-(2,6-dichloro-4-methyl-phenoxy)-ethoxy]-benzyl}-1-(8-fluoro-3,4-dihydro-2H-quinolin-1-yl)-propan-1-one;
3-Amino-2-{4-[2-(2,6-dichloro-4-methyl-phenoxy)-ethoxy]-benzyl}-1-piperidin-1-yl-propan-1-one;
3-Amino-2-{4-[2-(2,6-dichloro-4-methyl-phenoxy)-ethoxy]-benzyl}-1-(6-methoxy-3,4-dihydro-2H-quinolin-1-yl)-propan-1-one;
3-Amino-1-(6,8-dichloro-3,4-dihydro-2H-quinolin-1-yl)-2-{4-[2-(2,6-dichloro-4-methyl-phenoxy)-ethoxy]-benzyl}-propan-1-one;
3-Amino-2-{4-[2-(2,6-dichloro-4-methyl-phenoxy)-ethoxy]-benzyl}-1-(6-trifluoromethyl-3,4-dihydro-2H -quinolin-1-yl)-propan-1-one;
3-Amino-2-{4-[2-(2,6-dichloro-4-methyl-phenoxy)-ethoxy]-benzyl}-1-(6-trifluoromethyl-3,4-dihydro-2H -quinolin-1-yl)-propan-1-one hydrochloride;
3-Amino-2-{4-[2-(2,6-dichloro-4-methyl-phenoxy)-ethoxy]-benzyl}-1-(6-trifluoromethyl-3,4-dihydro-2H-quinolin-1-yl)-propan-1-one hemifumarate;
3-Amino-2-{4-[2-(2,6-dichloro-4-methyl-phenoxy)-ethoxy]-benzyl}-1-(6-methyl-3,4-dihydro-2H-quinolin-1-yl)-propan-1-one;
2-Aminomethyl-3-{4-[2-(2,6-dichloro-4-methyl-phenoxy)-ethoxy]-phenyl}-1-(6,7-dimethoxy-3,4-dihydro-1H-isoquinolin-2-yl)-propan-1-one;
3-Amino-2-{4-[2-(2,6-dichloro-4-methyl-phenoxy)-ethoxy]-benzyl}-1-(4-methyl-piperidin-1-yl)-propan-1-one;
3-Amino-2-{4-[2-(2,6-dichloro-4-methyl-phenoxy)-ethoxy]-benzyl}-1-(4-pyrimidin-2-yl-piperazin-1-yl)-propan-1-one;
3-Amino-2-{4-[2-(2,6-dichloro-4-methyl-phenoxy)-ethoxy]-benzyl}-1-(6-fluoro-3,4-dihydro-2H-quinolin-1-yl)-propan-1-one;
3-Amino-2-{4-[2-(2,6-dichloro-4-methyl-phenoxy)-ethoxy]-benzyl}-1-[4-(2-methoxy-ethoxy)-piperidin-1-yl]-propan-1-one;
2-Aminomethyl-3-{4-[2-(2,6-dichloro-4-methyl-phenoxy)-ethoxy]-phenyl}-1-(4-methyl-piperazin-1-yl)-propan-1-one;
3-Amino-2-{4-[2-(2,6-dichloro-4-methyl-phenoxy)-ethoxy]-benzyl}-1-pyrrolidin-1-yl-propan-1-one;
3-Amino-2-{4-[2-(2,6-dichloro-4-methyl-phenoxy)-ethoxy]-benzyl}-1-[6-(3-methoxy-propoxy)-3,4-dihydro-2H-quinolin-1-yl]-propan-1-one;
3-Amino-2-{4-[2-(2,6-dichloro-4-methyl-phenoxy)-ethoxy]-benzyl}-1-morpholin-4-yl-propan-1-one hydrochloride;
2-Aminomethyl-3-{4-[2-(2,6-dichloro-4-methyl-phenoxy)-ethoxy]-phenyl}-1-(3-methoxymethyl-piperidin-1-yl)-propan-1-one hydrochloride;
3-Amino-2-{4-[2-(2,6-dichloro-4-methyl-phenoxy)-ethoxy]-benzyl}-1-(6-trifluoromethoxy-3,4-dihydro-2H-quinolin-1-yl)-propan-1-one hydrochloride;
3-Amino-2-{4-[2-(2,6-dichloro-4-methyl-phenoxy)-ethoxy]-benzyl}-1-[6-(2,2,2-trifluoro-ethoxy)-3,4-dihydro-2H-quinolin-1-yl]-propan-1-one hydrochloride;
1-(2-Aminomethyl-3-{4-[2-(2,6-dichloro-4-methyl-phenoxy)-ethoxy]-phenyl}-propionyl)-pyrrolidine-2-carboxylic acid methyl ester hydrochloride;
2-Aminomethyl-3-{4-[2-(2,6-dichloro-4-methyl-phenoxy)-ethoxy]-phenyl}-1-(3-methoxy-piperidin-1-yl)-propan-1-one hydrochloride;
3-Amino-2-{4-[2-(2,6-dichloro-phenoxy)-ethoxy]-benzyl}-1-(6-trifluoromethyl-3,4-dihydro-2H-quinolin-1-yl)-propan-1-one hydrochloride;
3-Amino-2-{4-[2-(2,6-dichloro-phenoxy)-ethoxy]-benzyl}-1-(6-trifluoromethoxy-3,4-dihydro-2H-quinolin-1-yl)-propan-1-one hydrochloride;
3-Amino-2-{4-[2-(2-chloro-4-methyl-phenoxy)-ethoxy]-benzyl}-1-(6-trifluoromethyl-3,4-dihydro-2H-quinolin-1-yl)-propan-1-one;
3-Amino-2-{4-[2-(2,6-difluoro-phenoxy)-ethoxy]-benzyl}-1-(6-trifluoromethyl-3,4-dihydro-2H-quinolin-1-yl)-propan-1-one hydrochloride;
3-Amino-2-{4-[2-(2,6-difluoro-phenoxy)-ethoxy]-benzyl}-1-(6-fluoro-3,4-dihydro-2H-quinolin-1-yl)-propan-1-one hydrochloride;
3-Amino-2-{4-[2-(2,4-difluoro-phenoxy)-ethoxy]-benzyl}-1-(6-trifluoromethyl-3,4-dihydro-2H-quinolin-1-yl)-propan-1-one hydrochloride;
3-Amino-2-{4-[2-(2,4-dichloro-phenoxy)-ethoxy]-benzyl}-1-(6-fluoro-3,4-dihydro-2H-quinolin-1-yl)-propan-1-one hydrochloride;
3-Amino-2-{4-[2-(2,4-dichloro-phenoxy)-ethoxy]-benzyl}-1-(6-trifluoromethyl-3,4-dihydro-2H-quinolin-1-yl)-propan-1-one hydrochloride chloride;

3-Amino-2-{4-[2-(4-chloro-2-fluoro-phenoxy)-ethoxy]-benzyl}-1-(6-fluoro-3,4-dihydro-2H-quinolin-1-yl)-propan-1-one hydrochloride;
3-Amino-2-{4-[2-(4-chloro-2-fluoro-phenoxy)-ethoxy]-benzyl}-1-(6-trifluoromethyl-3,4-dihydro-2H-quinolin-1-yl)-propan-1-one hydrochloride;
3-Amino-2-{6-[2-(2,6-dichloro-4-methyl-phenoxy)-ethoxy]-pyridin-3-ylmethyl}-1-(6-trifluoromethyl-3,4-dihydro-2H-quinolin-1-yl)-propan-1-one;
2-Aminomethyl-3-{6-[2-(2,6-dichloro-4-methyl-phenoxy)-ethoxy]-pyridin-3-yl}-1-piperidin-1-yl-propan-1-one;
2-Aminomethyl-3-{6-[2-(2,6-dichloro-4-methyl-phenoxy)-ethoxy]-pyridin-3-yl}-1-(3,4-dihydro-2H-quinolin-1-yl)-propan-1-one;
3-Amino-2-{6-[2-(2,6-dichloro-4-methyl-phenoxy)-ethoxy]-pyridin-3-ylmethyl}-1-(6-fluoro-3,4-dihydro-2H-quinolin-1-yl)-propan-1-one hydrochloride;
2-Aminomethyl-3-{4-[3-(2,6-dichloro-4-methyl-phenoxy)-propoxy]-phenyl}-1-(3,4-dihydro-2H-quinolin-1-yl)-propan-1-one;
3-Amino-1-(6-fluoro-3,4-dihydro-2H-quinolin-1-yl)-2-{4-[3-(2-methoxy-benzyloxy)-propoxy]-benzyl}-propan-1-one;
3-Amino-2-{4-[3-(2-methoxy-benzyloxy)-propoxy]-benzyl}-1-(6-trifluoromethyl-3,4-dihydro-2H-quinolin-1-yl)-propan-1-one;
3-Amino-2-{4-[2-(2,6-dichloro-4-methyl-phenoxy)-ethoxy]-3-methoxy-benzyl}-1-(3,4-dihydro-2H-quinolin-1-yl)-propan-1-one hydrochloride;
1-(3-(6-Chloro-3,4-dihydro-2H-quinolin-1-yl)-2-{4-[2-(2,6-dichloro-4-methyl-phenoxy)-ethoxy]-benzyl}-3-oxo-propyl)-3-phenyl-urea;
[2-{4-[2-(2,6-Dichloro-4-methyl-phenoxy)-ethoxy]-benzyl}-3-oxo-3-(6-trifluoromethyl-3,4-dihydro-2H-quinolin-1-yl)-propyl]-carbamic acid tert-butyl ester;
3-Amino-2-{4-[2-(2,6-dichloro-phenyl)-ethoxy]-benzyl}-1-(6-trifluoromethyl-3,4-dihydro-2H-quinolin-1-yl)-propan-1-one hydrochloride;
3-Amino-2-{4-[2-(2,6-dichloro-phenyl)-ethoxy]-benzyl}-1-(6-fluoro-3,4-dihydro-2H-quinolin-1-yl)-propan-1-one hydrochloride;
3-Amino-1-(6-tert-butyl-3,4-dihydro-2H-quinolin-1-yl)-2-{4-[2-(2,6-dichloro-phenoxy)-ethoxy]-benzyl}-propan-1-one hydrochloride;
3-Amino-2-{4-[2-(piperidin-4-yloxy)-ethoxy]-benzyl}-1-(6-trifluoromethyl-3,4-dihydro-2H-quinolin-1-yl)-propan-1-one hydrochloride;
[1-(3-Amino-2-{4-[2-(2,6-dichloro-phenoxy)-ethoxy]-benzyl}-propionyl)-pyrrolidin-3-yl]-carbamic acid tert-butyl ester;
2-Aminomethyl-3-{4-[2-(2,6-dichloro-phenoxy)-ethoxy]-phenyl}-1-(2,3-dihydro-indol-1-yl)-propan-1-one hydrochloride;
3-Amino-2-{4-[2-(2,4-difluoro-phenoxy)-ethoxy]-benzyl}-1-(6-trifluoromethoxy-3,4-dihydro-2H-quinolin-1-yl)-propan-1-one hydrochloride;
3-Amino-2-{4-[2-(2,4-difluoro-phenoxy)-ethoxy]-benzyl}-1-piperidin-1-yl-propan-1-one hydrochloride;
3-Amino-2-{4-[2-(2,4-difluoro-phenoxy)-ethoxy]-benzyl}-1-(3,4-dihydro-2H-quinolin-1-yl)-propan-1-one hydrochloride;

Following compounds can be synthesized by following the procedure mentioned herein along with suitable modifications/alterations etc. which are within the scope of the person skilled in the art.
3-Amino-2-{4-[2-(2,4-difluoro-phenoxy)-ethoxy]-benzyl}-1-(2,3-dihydro-indol-1-yl)-propan-1-one hydrochloride;
3-Amino-2-{4-[2-(2,6-dichloro-phenoxy)-ethoxy]-benzyl}-1-(3-methoxymethyl-piperidin-1-yl)-propan-1-one hydrochloride;
3-Amino-2-{4-[2-(2,6-dichloro-phenoxy)-ethoxy]-benzyl}-1-piperidin-1-yl-propan-1-one hydrochloride;
1-(3-Amino-2-{4-[2-(2,6-dichloro-phenoxy)-ethoxy]-benzyl}-propionyl)-pyrrolidine-2-carboxylic acid methyl ester hydrochloride;
3-Amino-2-{4-[2-(2,6-dichloro-phenoxy)-ethoxy]-benzyl}-1-(3,4-dihydro-2H-quinolin-1-yl)-propan-1-one hydrochloride;
3-Amino-2-{4-[2-(2,6-dichloro-phenoxy)-ethoxy]-benzyl}-1-morpholin-4-yl-propan-1-one hydrochloride;
3-Amino-2-{4-[2-(2,6-dichloro-phenoxy)-ethoxy]-benzyl}-1-(6-methoxy-3,4-dihydro-2H-quinolin-1-yl)-propan-1-one hydrochloride;
3-Amino-2-{4-[2-(2,6-dichloro-phenoxy)-ethoxy]-benzyl}-1-pyrrolidin-1-yl-propan-1-one hydrochloride;
2-Aminomethyl-3-{6-[2-(2,6-dichloro-4-methyl-phenoxy)-ethoxy]-pyridin-3-yl}-1-(3-methoxymethyl-piperidin-1-yl)-propan-1-one hydrochloride;
2-Aminomethyl-3-{6-[2-(2,6-dichloro-phenoxy)-ethoxy]-pyridin-3-yl}-1-(3-methoxymethyl-piperidin-1-yl)-propan-1-one hydrochloride;
(R)-2-Aminomethyl-3-{4-[2-(2,6-dichloro-4-methyl-phenoxy)-ethoxy]-phenyl}-1-(2-methoxymethyl-pyrrolidin-1-yl)-propan-1-one hydrochloride;
(S)-2-Aminomethyl-3-{4-[2-(2,6-dichloro-4-methyl-phenoxy)-ethoxy]-phenyl}-1-(2-methoxymethyl-pyrrolidin-1-yl)-propan-1-one hydrochloride;

The compounds of the present invention may be prepared using the methods described below, together with conventional technique known to those skilled in the art of organic synthesis, or variation thereon as appreciated by those skilled in the art. Refereed method included, but not limited to those described below, where all symbols are define earlier.

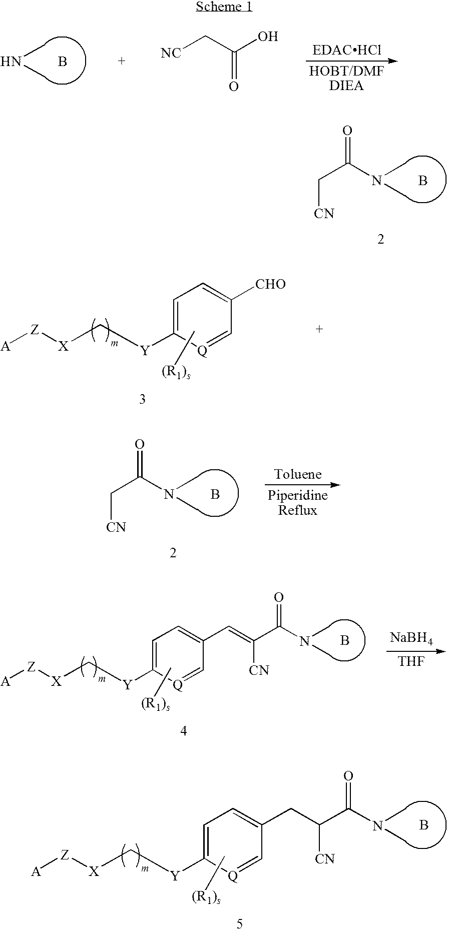

Propionitrile derivatives of formula (2), where all symbols are defined earlier, may be synthesized by reacting cyclic amine derivative (B), where all symbols are as described earlier with cyano acetic acid using carboxyl groups activating agents such as EDAC.HCl, DCC and the like in the presence of an additive like HOBT and base like triethyl amine or diisopropylethylamine in solvent(s) like dimethyl formamide or dichloromethane at temperature 0-25° C. Reacting aldehyde derivative of formula (3) with the propionitrile derivative of formula (2) as prepared above, in the presence of an organic base such as triethyl amine, piperidine, imidazole, ethyl diisopropyl amine and the like gives the acrylonitrile derivative of formula (4). The reaction may be performed at 25-110° C. and solvent(s) may be selected from benzene, toluene and the like or their suitable mixtures.

The reduction of double bond in acrylonitrile derivative of formula (4) as prepared above can be accomplished using hydrogenation or using suitable reducing agents like sodium borohydride, sodium cyanoborohydride, lithium aluminum hydride and the like to obtain nitrile derivative of formula (5). The reaction may be performed at −20 to +20° C. and suitable solvent(s) may be suitable ethereal solvents like diethyl ether, tetrahydrofuran and the like or halogenated solvent(s) like dichloromethane, dichloroethane and the like or suitable mixture thereof.

### Method A

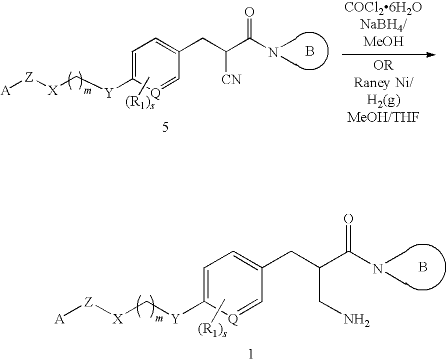

The reduction of cyano group in nitrile derivative of formula (5) as prepared earlier can be accomplished using suitable hydrogenation techniques or with suitable reducing agents such as cobalt(II) chloride hexahydrate-NaBH₄, nickel(II)chloride dihydrate-NaBH₄ and the like to get amine compound of formula (1). The reaction may be performed at −20 to +20° C. and solvents may be suitable ethereal solvent(s) like diethyl ether, tetrahydrofuran and the like or suitable protic solvent(s) like methanol, ethanol or mixture thereof.

### Method B

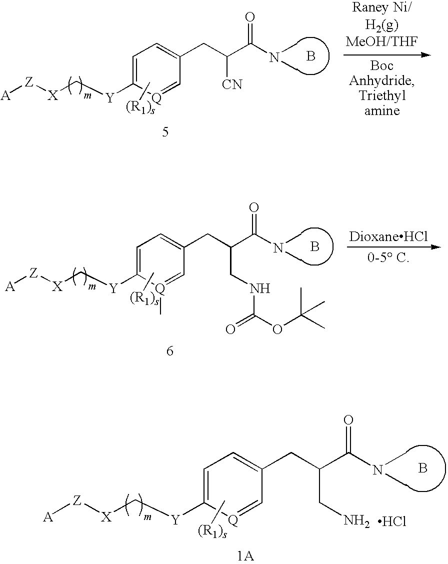

The reduction of cyano group in the nitrile derivative of formula (5) as prepared earlier can be accomplished using suitable hydrogenation techniques to get the corresponding amine derivatives and the protection of amine functionality so obtained, can be accomplished by adding suitable amine protecting groups like Boc anhydride to get protected amine compound of formula (6). The reaction may be performed at ambient temperature and solvents may be suitable ethereal solvent(s) like diethyl ether, tetrahydrofurane and like or suitable protic solvent(s) like methanol, ethanol or their suitable mixtures. Removal of amine protecting group in compound (6) can be accomplished using suitable amino deprotecting agents like dioxane.HCl, methanolic.HCl, DCM-TFA and like to afford amine hydrochloride compound (1A). The reaction may be performed at 0 to 25° C.

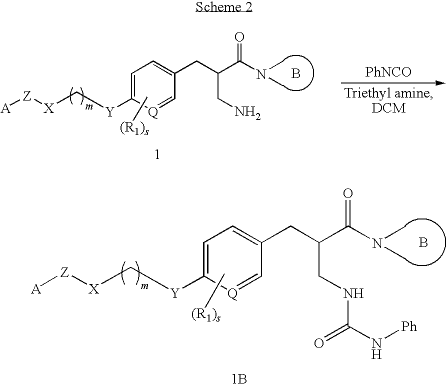

Urea derivative (1B), where all symbols are defined earlier, may be synthesized by reacting amine derivative (1), where all the symbols are define earlier, with an appropriate isocyanate in chlorinating solvent(s) like dichloro methane, dichloro ethane and like. The base used may be any suitable base(s) such as triethylamine, diisopropyl ethyl amine and like. The reaction may be performed at temperature 0 to 40° C.

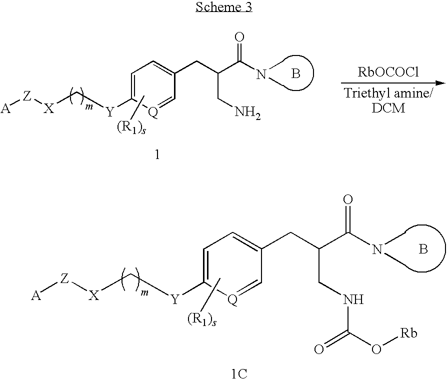

Carbamate derivatives (1C), where all the symbols define earlier, may be synthesized by reacting amine derivative (1), where all the symbols define earlier, with appropriate alkyl or aryl chloro formates in the presence of suitable base(s) like triethyl amine, diisopropyl ethyl amine and like, using chlorinating solvent(s) like dichloromethane and the like or dimethyl formamide. The reaction may be performed at 0 to 100° C.

The pharmaceutically acceptable salts forming a part of this invention may be prepared by treating the compound of formula (1) with suitable acids in suitable solvents by processes known in the art.

It will be appreciated that in any of the above mentioned reactions any reactive group in the substrate molecule may be protected, according to conventional chemical practice. Suitable protecting group in any of the above mentioned reactions are those used conventionally in the art. The methods of formation and removal of such protecting groups are those conventional methods appropriate to the molecule being protected. T. W. Greene and P. G. M. Wits “Protective groups in Organic Synthesis”, John Wiley & Sons, Inc, 1999, 3^{rd} Ed., 201-245 along with references therein gives such conventional methods and are incorporated herein as references.

The novel compounds of the present invention can be formulated into suitable pharmaceutically acceptable compositions by combining with suitable excipients by technique and processes and concentrations as are well known.

The compounds of formula (1) or pharmaceutical compositions containing them are useful as Renin inhibitors suitable for humans and other warm blooded animals, and may be administered either by oral, topical or parenteral administration.

The pharmaceutical composition is provided by employing conventional techniques. Preferably the composition is in unit dosage form containing an effective amount of the active component, that is, the compounds of formula (1) according to this invention.

The quantity of active component optionally substituted, that is the compounds of formula (1) according to this invention, in the pharmaceutical compositions and unit dosage form thereof may be varied or adjusted widely depending upon the particular application method, the potency of the particular compound and the desired concentration. Generally, the quantity of active component will range from 0.5% to 90% by weight of the composition.

The compounds of the present invention are suitable as Renin inhibitors and are useful in the treatment of hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephrtis renal colic, complication resulting from diabetes such as nephropathy, vasculopathy, and neuropathy, glaucoma, elevated intraocular pressure, atherosclerosis, restenosis post angioplasty, complications following vascular or cardiac surgery, erectile dysfunction.

The invention is further exemplified by the following examples below, which provides one of the several preferred embodiments of the present invention. These examples are provided merely as representative embodiments and should not be construed to limit the scope of the invention in any way.

### Preparation:

The following Aldehyde building blocks were synthesized by the process described beneath:

| Compound | Name |
|---|---|
| Aldehyde 1 | 4-[2-(2,6-Dichloro-4-methyl-phenoxy)-ethoxy]- |
| | benzaldehyde |
| Aldehyde 2 | 4-[2-(2,5-Dimethyl-phenoxy)-ethoxy]-benzaldehyde |
| Aldehyde 3 | 4-[2-(2,6-Dichloro-phenoxy)-ethoxy]-benzaldehyde |
| Aldehyde 4 | 4-[2-(2,6-Difluoro-phenoxy)-ethoxy]-benzaldehyde |
| Aldehyde 5 | 4-[2-(2,4-Difluoro-phenoxy)-ethoxy]-benzaldehyde |
| Aldehyde 6 | 4-[2-(2,4-Dichloro-phenoxy)-ethoxy]-benzaldehyde |
| Aldehyde 7 | 4-[2-(4-Chloro-2-fluoro-phenoxy)-ethoxy]-benzaldehyde |
| Aldehyde 8 | 6-[2-(2,6-Dichloro-4-methyl-phenoxy)-ethoxy]-pyridine-3- |
| | carbaldehyde |
| Aldehyde 9 | 4-[3-(2,6-Dichloro-4-methyl-phenoxy)-propoxy]- |
| | benzaldehyde |
| Aldehyde 10 | 4-[3-(2-Methoxy-benzyloxy)-propoxy]-benzaldehyde |
| Aldehyde 11 | 4-[2-(2,6-Dichloro-4-methyl-phenoxy)-ethoxy]-3-methoxy- |
| | benzaldehyde |
| Aldehyde 12 | 4-[2-(2-Chloro-4-methyl-phenoxy)-ethoxy]-benzaldehyde |
| Aldehyde 13 | 4-[2-(2,6-Dichloro-phenyl)-ethoxy]-benzaldehyde |
| Aldehyde 14 | 4-[2-(4-Formyl-phenoxy)-ethoxy]-piperidine-1-carboxylic |
| | acid tert-butyl ester |

### Aldehyde 1: 4-[2-(2,6-Dichloro-4-methyl-phenoxy)-ethoxy]-benzaldehyde

### Step 1: 2-(2,6-Dichloro-4-methyl-phenoxy)-ethanol

2,6-Dichloro-4-methyl phenol (1 eq), ethylene carbonate (1.5 eq) and piperidine (0.1 eq) were combined and heated at 140° C. for 6 h to afford the title compound as brown oil.

### Step 2: Aldehyde 1

2-(2,6-Dichloro-4-methyl-phenoxy)-ethanol (1 eq) obtained from step 1 above, and 4-hydroxy benzaldehyde (1 eq) were taken up in 10 v of dry toluene. To this, was then added triphenyl phosphine (1.3 eq) and finally diisopropylazadicarboxylate (1.5 eq) at 0-10° C.

Reaction mixture was allowed to attain room temperature and stirred for 14-18 h. Mixture was diluted with water and compound was extracted with EtOAc. Combined organic layer was washed with brine and dried over sodium sulfate. Filtration and concentration of filtrate in vacuo afforded a yellow semi solid. Purification of the crude product thus obtained by way of flash column chromatography (SiO₂ 4:1 (v/v) Hexane:EtOAc) afforded the title compound as white needle.

### Aldehyde 2: 4-[2-(2,5-Dimethyl-phenoxy)-ethoxy]-benzaldehyde

Prepared similar to the procedure described in Aldehyde 1 but using instead 2,5-dimethyl phenol as starting material.

### Aldehyde 3: 4-[2-(2,6-Dichloro-phenoxy)-ethoxy]-benzaldehyde

Prepared similar to the procedure described in Aldehyde 1 but using instead 2,6-dichloro phenol as starting material.

### Aldehyde 4: 4-[2-(2,6-Difluoro-phenoxy)-ethoxy]-benzaldehyde

Prepared similar to the procedure described in Aldehyde 1 but using instead 2,6-difluoro phenol as starting material.

### Aldehyde 5: 4-[2-(2,4-Difluoro-phenoxy)-ethoxy]-benzaldehyde

Prepared similar to the procedure described in Aldehyde 1 but using instead 2,4-difluoro phenol as starting material.

### Aldehyde 6: 4-[2-(2,4-Dichloro-phenoxy)-ethoxy]-benzaldehyde

Prepared similar to the procedure described in Aldehyde 1 but using instead 2,4-dichloro phenol as starting material.

### Aldehyde 7: 4-[2-(4-Chloro-2-fluoro-phenoxy)-ethoxy]-benzaldehyde

Prepared similar to the procedure described in Aldehyde 1 but using instead 2-fluoro-4-chloro phenol as starting material.

### Aldehyde 8: 6-[2-(2,6-Dichloro-4-methyl-phenoxy)-ethoxy]-pyridine-3-carbaldehyde

### Step 1: 5-Bromo-2-[2-(2,6-dichloro-4-methyl-phenoxy)-ethoxy]-pyridine

To a solution of 2-(2,6-dichloro-4-methyl-phenoxy)-ethanol (1 eq) in dry THF, sodium hydride (3.5 eq) was added at 0° C. Mixture was allowed to warm to room temperature and stirred for 1 h. To this, 2,5-dibromo pyridine (1 eq) was added dropwise and mixture was heated at 90° C. for 2 h. Organic volatiles were removed under reduced pressure. Mixture was diluted with EtOAc. Organic layer was washed with water, brine, dried over sodium sulfate and evaporated under reduced pressure to afford title compound as white solid.

### Step 2: Aldehyde 8

n-BuLi (1.15 eq) was added to the mixture of 5-bromo-2-[2-(2,6-dichloro-4-methyl-phenoxy)-ethoxy]-pyridine in THF at −40° C. The mixture was stirred for 1-3 h at same temperature and DMF (3 eq) was added. Mixture was stirred for 2-5 h at −40° C. Mixture was allowed to warm to room temperature and stirred at 14-18 h. Mixture was quenched in saturated NH₄Cl solution and extracted with diethyl ether. Organic layer was washed with brine dried over sodium sulfate, filtered and evaporated in vacuo afforded semi solid. Purification of the crude product thus obtained by the way of flash column chromatography (SiO₂, 7:3 (v/v) hexane:EtOAc) afforded title compound as white solid.

### Aldehyde 9: 4-[3-(2,6-Dichloro-4-methyl-phenoxy)-propoxy]-benzaldehyde

### Step 1: 3-(2,6-Dichloro-4-methyl-phenoxy)-propan-1-ol

2,6-Dichloro-4-methyl phenol (1 eq), 3-bromo-1-propanol (1.2 eq) and anhydrous K₂CO₃ (2.5 eq) were mixed in dry acetone (30 v) and heated to refluxed for 4-8 h.

Mixture was quenched in water. Compound was extracted with EtOAc. Combined organic layer was washed with brine, dried over sodium sulfate and evaporated in vacuo to afforded title compound as brown liquid.

### Step 2: Aldehyde 9

3-(2,6-Dichloro-4-methyl-phenoxy)-propan-1-ol (1 eq), 4-hydroxy benzaldehyde (1 eq) were taken in dry toluene (10 v). Triphenyl phosphine (1.3 eq) and diisopropylazadicarboxylate (1.5 eq) was added to this mixture at 0-10° C. Reaction mixture was allowed to attained room temperature and stirred for 14-18 h. Mixture was quenched in water and extracted with EtOAc. Combined organic layer was washed with brine, dried over sodium sulfate, filtered and evaporated in vacuo to afford semi solid compound. Purification of the crude product thus obtained by the way of flash column chromatography (SiO₂, 4:1 (v/v) Hexane:EtOAc) afforded the title compound as white solid.

### Aldehyde 10: 4-[3-(2-Methoxy-benzyloxy)-propoxy]-benzaldehyde

### Step 1: 3-(2-Methoxy-benzyloxy)-propan-1-ol

2-Methoxy benzyl alcohol (1 eq) was taken in dry DMF and sodium hydride (50%, 2.2 eq) was added to it at 0° C. Mixture was stirred at 25° C. for 1 hr and 3-bromo-1-propanol (1.2 eq) was added to the mixture at 0° C. Mixture was stirred at 25° C. for 2 h and quenched in water. Product was extracted with diethyl ether. Organic layer was washed with water, dried over sodium sulfate, filtered and evaporated in vacuo to afforded oily compound. Purification of crude product thus obtained by the way of flash column chromatography (SiO₂, 7:3 (v/v) hexane:EtOAc) to afford title compound as colorless oil.

### Step 2: Aldehyde 10

2-Methoxy-benzyloxy)-propan-1-ol (1 eq), 4-hydroxy benzaldehyde (1 eq) and triphenyl phosphine (1.2 eq) were taken in dry toluene (10 v). Diisopropyl azadi carboxylate (1.5 eq) was added to this mixture at 0° C. Mixture was warmed to room temperature and stirred for 14-18 h. Mixture was quenched in water. Organic layer was separated and product was extracted with EtOAc. Combined organic layer was washed with brine, dried over sodium sulfate, filtered and evaporated in vacuo to afforded crude semisolid. Purification of the crude product thus obtained by the way of flash column chromatography (SiO₂, 3:7 (v/v) Hexane:EtOAc) to afford title compound as off white solid.

### Aldehyde 11: 4-[2-(2,6-Dichloro-4-methyl-phenoxy)-ethoxy]-3-methoxy-benzaldehyde

Prepared similar to the procedure described in Aldehyde 1 but using instead 3-methoxy-4-hydroxy benzaldehyde in step 2.

### Aldehyde 12: 4-[2-(2-Chloro-4-methyl-phenoxy)-ethoxy]-benzaldehyde

Prepared similar to the procedure described in Aldehyde 1 but using instead 2-chloro-4-methyl phenol as starting material.

### Aldehyde 13: 4-[2-(2,6-Dichloro-phenyl)-ethoxy]-benzaldehyde

### Step 1: 2-(2,6-Dichloro-phenyl)-ethanol

2,6-Dichloro phenyl acetic acid methyl ester (1 eq) was taken in THF:Water (10:0.1 (v/v) and cooled to 5-10° C. NaBH₄ was added to this mixture and mixture was stirred for 4 h. Mixture was quenched in water and extracted with EtOAc. Organic layer was washed with brine and evaporated in vacuo to afford title compound as thick liquid.

### Step 2: Aldehyde 13

Prepared similar to the procedure described in Aldehyde 1, Step 2 but using instead 2-(2,6-dichloro-phenyl)-ethanol as starting material.

### Aldehyde 14: 4-[2-(4-Formyl-phenoxy)-ethoxy]-piperidine-1-carboxylic acid tert-butyl ester

Prepared similar to the procedure described in Aldehyde 1, Step 2 but using instead 4-(2-hydroxy-ethoxy)-piperidine-1-carboxylic acid tert-butyl ester as starting material.

The cyclic amine building blocks in Table 2 were synthesized as follow

TABLE 2

| Compound | Name |
|---|---|
| Amine 1 | 6-Fluoro-1,2,3,4-tetrahydro quinoline |
| Amine 2 | 6-Chloro-1,2,3,4-tetrahydro quinoline |
| Amine 3 | 7,8-Dichloro-1,2,3,4,-tetrahydro quinoline |
| Amine 4 | 8-Methoxy 1,2,3,4-tetrahydro quinoline |
| Amine 5 | 7,8-Dimethyl-1,2,3,4-tetrahydro quinoline |
| Amine 6 | 6-Methyl-1,2,3,4-tetrahydro quinoline |
| Amine 7 | 8-Fluoro-1,2,3,4-tetrahydro quinoline |
| Amine 8 | 6-Trifluoromethyl-1,2,3,4-tetrahydro quinoline |
| Amine 9 | 6,8-Dichloro-1,2,3,4-tetrahydroquinoline |
| Amine 10 | 6-Trifluomethoxy-1,2,3,4-tetrahydro quinoline |
| Amine 11 | 6-Bromo-1,2,3,4-tetrahydro quinoline |
| Amine 12 | 6-(2,2,2-Trifluoro-ethoxy)-1,2,3,4-tetrahydro-quinoline |
| | hydrochloride |
| Amine 13 | 6-(3-Methoxy-propoxy)-1,2,3,4-tetrahydro-quinoline |
| | hydrochloride |
| Amien 14 | 3-Methoxymethyl-piperidine hydrochloride |
| Amine 15 | 3-Methoxy-piperidine hydrochloride |
| Amine 16 | 4-(2-Methoxy-ethoxy)-piperidine hydrochloride |
| Amine 17 | 6-tert-Butyl-1,2,3,4-tetrahydro quinoline |

### Amine 1: 6-Fluoro-1,2,3,4-tetrahydro-quinoline

### Step 1: 6-Fluoro quinoline

Concentrated sulfuric acid (3 eq) was added dropwise to a vigorously stirred mixture of 4-fluoro aniline (1 eq), 1₂ (0.1 eq), in glycerol (1.5 eq) within 0.5 h, wherein the temperature of the mixture rises to 65-70° C. The mixture was then heated to 135-140° C. for 10-12 h giving dark brown forming mixture. The mixture was cooled to room temperature and quenched in ice cooled water. pH of the solution was adjusted to 8 to 9 by adding 25-30% ammonia solution. Aqueous layer was extracted with EtOAc. Organic layer was washed with brine, dried over sodium sulfate and evaporated in vacuo to get crude product. Purification of the crude product thus obtained by the way of flash column chromatography (SiO₂, 8:2 (v/v) hexane:EtOAc) to afforded title compound as dark red liquid.

### Step 2: Amine 1

Sodium cyanoborohydride (3 eq) was added gradually to the solution of 6-fluoro quinoline (1 eq) in glacial acetic acid (3 v) at ambient temperature. After stirring for 6 h the reaction mixture was quenched in water and extracted with EtOAc. The combined organic layers were washed with water, brine and dried over sodium sulfate, filtered and evaporated in vacuo to get crude product. The desired product was purified by column chromatography (SiO₂, 3:7 hexane:EtOAc) afforded title compound as light yellow liquid.

### Amine 2: 6-Fluoro-1,2,3,4-tetrahydro-quinoline

Prepared similar to the procedure described in amine 1 but using instead 4-chloro aniline as starting material.

### Amine 3: 7,8-Dichloro-1,2,3,4-tetrahydro-quinoline

Prepared similar to the procedure described in amine 1 but using instead 2,3-dichloro aniline as starting material.

### Amine 4: 8-Methoxy-1,2,3,4-tetrahydroquinoline

Prepared similar to the procedure described in amine 1 but using instead 2-methoxy aniline as starting material.

### Amine 5: 7,8-Dimethyl-1,2,3,4-tetrahydro quinoline

Prepared similar to the procedure described in amine 1 but using instead 2,3-dimethyl aniline as starting material.

### Amine 6: 6-Methyl-1,2,3,4-tetrahydroquinoline

Prepared similar to the procedure described in amine 1 but using instead 4-methyl quinoline as starting material.

### Amine 7: 8-Fluoro-1,2,3,4-tetrahydro quinoline

Prepared similar to the procedure described in amine 1 but using instead 2-fluoro quinoline as starting material.

### Amine 8: 6-Trifluoromethyl-1,2,3,4-tetrahydroquinoline

Prepared similar to the procedure described in amine 1 but using instead 4-trifluoromethylaniline as starting material.

### Amine 9: 6,8-Dichloro-1,2,3,4-tetrahydro quinoline

Prepared similar to the procedure described in amine 1 but using instead 2,4-dichloro aniline as starting material.

### Amine 10: 6-Trifluoromethoxy-1,2,3,4-tetrahydro quinoline

Prepared similar to the procedure described in amine 1 but using instead 4-trifluoromethoxy aniline as starting material.

### Amine 11: 6-Bromo-1,2,3,4-tetrahydro quinoline

Prepared similar to the procedure described in amine 1 but using instead 4-bromo aniline as starting material.

### Amine 12: 6-(2,2,2-Trifluoro-ethoxy)-1,2,3,4-tetrahydro-quinoline hydrochloride

### Step 1: 6-Hydroxy-3,4-dihydro-2H-quinoline-1-carboxylic acid tert-butyl ester

6-Hydroxy-1,2,3,4-tetrahydro quinoline (1 eq), Boc anhydride (1.2 eq) and triethyl amine (1.2 eq) were mixed and heated to refluxed for 2 hr. Mixture was quenched in water and product was extracted with EtOAc to afford title compound as oil. Compound was used directly for the next reaction without purification.

### Step 2: 6-(2,2,2-Trifluoro-ethoxy)-3,4-dihydro-2H-quinoline-1-carboxylic acid tert-butyl ester

6-(2,2,2-Trifluoro-ethoxy)-1,2,3,4-tetrahydro-quinoline (1 eq), methanesulfonic acid 2,2,2-trifluoro-ethyl ester (1 eq) and anhydrous K₂CO₃ were mixed in DMF (5 v) and mixture was heated to 80-100° C. Reaction mixture was quenched in water. Product was extracted with EtOAc; organic layer was washed with brine, dried over sodium sulfate, filtered and evaporated in vacuo to get brown color oily compound.

### Step 3: Amine 12

Dioxane.HCl (10%, 2v) was added to 6-(2,2,2-trifluoro-ethoxy)-3,4-dihydro-2H-quinoline-1-carboxylic acid tert-butyl ester (1 eq) and mixture was stirred for 1 h at 0-5° C. Organic volatiles were removed under reduced pressure to afford title compound as brown liquid.

### Amine 13: 6-(3-Methoxy-propoxy)-1,2,3,4-tetrahydro-quinoline hydrochloride

### Step 1: 6-Hydroxy-3,4-dihydro-2H-quinoline-1-carboxylic acid tert-butyl ester

Prepared as per the procedure reported in Amine 12 Step 1

### Step 2: 6-(3-Methoxy-propoxy)-3,4-dihydro-2H-quinoline-1-carboxylic acid tert-butyl ester

6-Hydroxy-3,4-dihydro-2H-quinoline-1-carboxylic acid tert-butyl ester (1 eq) obtained from step 1, 1-bromo-3-methoxy-propane (1.1 eq) and anhydrous K₂CO₃ (1.5 eq) were mixed in DMF (5 v) and heated to 70-80° C. for 2-5 h. Mixture was quenched in water and product was extracted with EtOAc. Combined organic layers were washed with brine, dried over sodium sulfate, filtered and evaporated in vacuo to afford title compound as oil. The crude product was purified by the way of flash column chromatography (SiO₂, 3:7 (v/v)::Hexane:EtOAc) to afford title compound as light brown liquid.

### Step 3: Amine 13

Dioxane.HCl (10%, 2v) was added to 6-(3-methoxy-propoxy)-3,4-dihydro-2H-quinoline-1-carboxylic acid tert-butyl ester (1 eq) and mixture was stirred for 1 h at 0-5° C. Organic volatiles were removed under reduced pressure to afford title compound as brown syrupy liquid.

### Amine 14: 3-Methoxymethyl-piperidine hydrochloride

### Step 1: 3-Methanesulfonyloxymethyl-piperidine-1-carboxylic acid tert-butyl ester

N-Boc-3-hydroxymethyl piperidine (1 eq), triethylamine (2 eq) were taken in DCM (10 v). Methane sulfonyl chloride (1.2 eq) was added to the mixture at 0° C. Mixture was stirred at 10° C. for 2-5 h. Reaction mixture was quenched in water. Organic layer was separated washed with brine, dried over sodium sulfate, filtered, and evaporated in vacuo to get title compound as semi solid.

### Step 2: 3-Methoxymethyl-piperidine-1-carboxylic acid tert-butyl ester

Sodium metal (3 eq) was dissolved in dry methanol (10 v) and 3-methanesulfonyloxy methyl-piperidine-1-carboxylic acid tert-butyl ester (1 eq) was added to this stirred solution at 10° C. Mixture was refluxed form 3 h. Mixture was quenched in water. Product was extracted by EtOAc. Organic layer was washed with water, brine, dried over sodium sulfate, filtered and evaporated in vacuo to afforded title compound as liquid.

### Step 3: Amine 14

Dioxane.HCl (10%) was added to 3-methoxymethyl-piperidine-1-carboxylic acid tert-butyl ester (1 eq) and mixture was stirred for 1 h at 0-5° C. Organic volatiles were removed under reduced pressure to afford title compound as yellow solid. Amine 15: 3-Methoxy-piperidine hydrochloride

### Step 1: 3-Methoxy-piperidine-1-carboxylic acid tert-butyl ester

N-Boc-3-hydroxy piperidine (1 eq) was dissolved in THF (5 v), to this sodium hydride (50%, 2.2 eq) was added at 0° C. Mixture was stirred for lh and methyl iodide (1.2 eq) was added. Mixture was stirred for 2-6 h and diluted with water. Product was extracted with EtOAc. Organic layer was washed with water, brine, dried over sodium sulfate, filtered and evaporated in vacuo to afford title compound as thick liquid.

### Step 2: Amine 15

Prepared similar to the procedure described in Amine 14 step 3 but using instead 3-methoxy-piperidine-1-carboxylic acid tert-butyl ester as starting material.

### Amine 16: 4-(2-Methoxy-ethoxy)-piperidine hydrochloride

### Step 1: 4-tert-Butoxycarbonylmethoxy-piperidine-1-carboxylic acid tert-butyl ester

N-Boc-4-hydroxy piperidine (1 eq), tetrabutyl ammonium hydrogen sulfate (0.3 eq) and sodium hydroxide (1.2 eq) were taken in toluene and bromo-acetic acid tert-butyl ester (1.8 eq) was added to this mixture. Mixture was allowed to reflux for 4 h. Mixture was quenched in water, organic layer was separated, dried over sodium sulfate, filtered and evaporated in vacuo to afford title compound as off white solid.

### Step 2: 4-(2-Hydroxy-ethoxy)-piperidine-1-carboxylic acid tert-butyl ester

4-tert-Butoxycarbonylmethoxy-piperidine-1-carboxylic acid tert-butyl ester (1 eq) was dissolved in dry THF (5 v), to this LiALH₄ (1.2 eq) was added at 0-5° C. and mixture was stirred for 2 h. Mixture was quenched in saturated ammonium chloride solution. Product was extracted with EtOAc. Combined organic layer was washed with brine, dried over sodium sulfate, filtered and evaporated in vacuo to afford title compound as liquid. Crude product obtained was used directly for the next reaction without further purifications.

### Step 3: 4-(2-Methoxy-ethoxy)-piperidine-1-carboxylic acid tert-butyl ester

4-(2-Hydroxy-ethoxy)-piperidine-1-carboxylic acid tert-butyl ester (1 eq) was dissolved in DMF (5v), to this NaH (50%, 2.2 eq) was added to this mixture at 5-10° C. followed by methyl iodide (1.2 eq). Mixture was stirred for 2 h at 25° C. and quenched in water. Product was extracted with EtOAc. Organic layer was washed with brine, dried over sodium sulfate, filtered and evaporated in vacuo to afford title compound as thick liquid.

### Step 4: Amine 16

Prepared similar to the procedure described in Amine 14 step 3 but using instead 4-(2-methoxy-ethoxy)-piperidine-1-carboxylic acid tert-butyl ester as starting material.

### Amine 17: 6-tert-butyl-1,2,3,4-tetrahydroquinoline

Prepared similar to the procedure described in amine 1 but using instead 4-tert-butyl aniline as starting material

HPLC Condition.

HPLC column: ODS-(150×4.6)4 u, C-18

Mobile phase: 0.05% TFA buffer: ACN (Gradient)

Flow rate: 1.0 mL/min

Wavelength: UV at 220 nm

### Example 1

### Preparation of 2-Aminomethyl-1-(3,4-dihydro-2H-quinolin-1-yl)-3-{4-[2-(2,5-dimethyl-phenoxy)-ethoxy]-phenyl}-propan-1-one

### Step 1: Preparation of 3-(3,4-dihydro-2H-quinolin-1-yl)-3-oxo-propionitrile

To a solution of cyano acetic acid [1 eq] in DMF [5v], was added HOBT [1.3 eq]. To this reaction mixture was added EDAC.HCl [1.2 eq], 1,2,3,4-tetrahydroquinoline [1 eq] and diisopropyl ethylamine [3 eq] under N₂ at 0-5° C. The resulting reaction mixture was stirred at 25° C. for 14-18 h. Mixture was quenched in water. The aqueous layer was extracted with EtOAc. The organic layer was washed with brine, dried over sodium sulfate and concentrated in vacuo to afford light yellow liquid. Purification of crude product was thus obtained by the way of column chromatography (SiO₂, Hexane to 1:10 (v/v) EtOAc:hexane) to get sticky solid. The title compound was characterized by spectral analysis. ESI-MS 200.8 (M+H)⁺

### Step 2: 2-(3,4-dihydro-2H-quinoline-1-carbonyl)-3-{4-[2-(2,5-dimethyl-phenoxy)-ethoxy]-phenyl}-acrylonitrile

4-[2-(2,5-Dimethyl-phenoxy)-ethoxy]-benzaldehyde [1 eq] and 3-(3,4-dihydro-2H-quinolin-1-yl)-3-oxo-propionitrile [1 eq] obtained from step 1, was dissolved in toluene. To this solution was added few drops of piperidine. Dean-Stark apparatus was attached to the reaction assembly. The resulting pale yellow solution was heated to reflux for 5 h and volatiles were removed in vacuo. The semi solid mass obtained was washed with diisopropyl ether. The title compound was isolated as light yellow solid. The compound was characterized by spectral analysis. ESI-MS 453 (M+H)⁺

### Step 3: Preparation of 3-(3,4-dihydro-2H-quinolin-1-v1)-2-{4-[2-(2,5-dimethyl-phenoxy)-ethoxy]-benzyl}-3-oxo-propionitrile

2-(3,4-Dihydro-2H-quinoline-1-carbonyl)-3-{4-[2-(2,5-dimethyl-phenoxy)-ethoxy]-phenyl}-acrylonitrile [1 eq] obtained from step 2 was dissolved in 10:1 (v/v) THF:water. Sodium borohydride [6 eq] was added at 0° C. The reaction mixture was stirred at 0° C. for 2-4 hr. Reaction mixture was quenched in water. The aqueous layer was extracted with EtOAc. The organic layer was washed with brine, dried over sodium sulfate and concentrated in vacuo to afford colorless oily compound. The title compound was characterized by spectral analysis. ESI-MS 455 (M+H)⁺

### Step 4: 2-Aminomethyl-1-(3,4-dihydro-2H-quinolin-1-yl)-3-{4-[2-(2,5-dimethylphenoxy)-ethoxy]-phenyl}-propan-1-one

This compound was prepared using the general process described in Method-A above. To a solution of 3-(3,4-dihydro-2H-quinolin-1-yl)-2-{4-[2-(2,5-dimethyl-phenoxy)-ethoxy]-benzyl}-3-oxo-propionitrile [1 eq] obtained from step 3 and cobalt(II) chloride hexahydrate [2 eq] in methanol, sodium borohydride [1 eq] was added at 0° C. in parts. The mixture was stirred for 3 hr at 0° C. The mixture was quenched in water; aqueous layer was extracted with dichloromethane. Organic layer was washed with brine, dried over sodium sulfate and concentrated in vacuo to afford thick liquid. Purification of crude product was thus obtained by the way of column chromatography (Aluminium oxide Basic 0.1:10 (v/v)::chloroform:methanol). The title compound was isolated as thick liquid and was characterized by spectral analysis. ESI-MS 459(M+H)⁺ HPLC t_{Ret}; 17.07 min.

The following compounds (Example 2-24) were prepared by following the general process described in Method-A above, at appropriate places as in Example 1.

### Example 2

### 3-Amino-2-{4-[2-(2,6-dichloro-4-methyl-phenoxy)-ethoxy]-benzyl}-1-(3,4-dihydro-2H-quinolin-1-yl)-propan-1-one

Prepared similar to the procedure described in Example 1 but using instead Aldehyde 2 and 1,2,3,4-tetrahydro quinoline as starting material. The title compound was obtained as thick liquid ESI-MS: M⁺=513.1; HPLC t_{Ret}; 17.27 min.

### Example 3

### 3-Amino-2-{4-[2-(2,6-dichloro-4-methyl-phenoxy)-ethoxy]-benzyl}-1-(3,4-dihydro-1H-isoquinolin-2-yl)-propan-1-one

Prepared similar to the procedure described in Example 1 but using instead Aldehyde 2 and 1,2,3,4-tetrahydroisoquinoline. The title compound was obtained as thick liquid ESI-MS: M⁺: 513.0; HPLC t_{Ret}; 17.06 min.

### Example 4

### 3-Amino-2-{4-[2-(2,6-dichloro-4-methyl-phenoxy)-ethoxy]-benzyl}-1-(2,3-dihydro-benzo[1,4]oxazin-4-yl)-propan-1-one

Prepared similar to the procedure described in Example 1 but using instead Aldehyde 2 and 3,4-Dihydro-2H-benzo[1,4]oxazine. The title compound was obtained as thick liquid ESI-MS: m⁺: 515.0; HPLC t_{Ret}; 17.06 min.

### Example 5

### 3-Amino-1-(6-chloro-3,4-dihydro-2H-quinolin-1-yl)-2-{4-[2-(2,6-dichloro-4-methyl-phenoxy)-ethoxy]-benzyl}-propan-1-one

Prepared similar to the procedure described in Example 1 but using instead Aldehyde 2 and Amine 2. The title compound was obtained as thick liquid ESI-MS: M⁺: 548.70; HPLC t_{Ret}: 17.91 min.

### Example 6

### 3-Amino-1-(6-bromo-3,4-dihydro-2H-quinolin-1-yl)-2-{4-[2-(2,6-dichloro-4-methyl-phenoxy)-ethoxy]-benzyl}-propan-1-one

Prepared similar to the procedure described in Example 1 but using instead Aldehyde 2 and Amine 11. The title compound was obtained as thick liquid ESI-MS: M⁺: 593.0; HPLC t_{Ret}: 18.01 min.

### Example 7

### 3-Amino-1-(7,8-dichloro-3,4-dihydro-2H-quinolin-1-yl)-2-{4-[2-(2,6-dichloro-4-methyl-phenoxy)-ethoxy]-benzyl}-propan-1-one

Prepared similar to the procedure describe in Example 1 but using instead Aldehyde 2 and Amine 3. The title compound was obtained as thick liquid ESI-MS: M⁺: 582.8; HPLC t_{Ret}: 18.08 min.

### Example 8

### 3-Amino-2-{4-[2-(2,6-dichloro-4-methyl-phenoxy)-ethoxy]-benzyl}-1-(8-methoxy-3,4-dihydro-2H-quinolin-1-yl)-propan-1-one

Prepared similar to the procedure described in Example 1 but using instead Aldehyde 2 and Amine 4. The title compound was obtained as thick liquid ESI-MS: M⁺: 543.0; HPLC t_{Ret}: 17.46 min

### Example 9

### 3-Amino-2-{4-[2-(2,6-dichloro-4-methyl-phenoxy)-ethoxy]-benzyl}-1-(7,8-dimethyl-3,4-dihydro-2H-quinolin-1-yl)-propan-1-one

Prepared similar to the procedure described in Example 1 but using instead Aldehyde 2 and Amine 5. The title compound was obtained as thick liquid ESI-MS: M⁺: 541.0; HPLC t_{Ret}: 18.16 min

### Example 10

### 3-Amino-2-{4-[2-(2,6-dichloro-4-methyl-phenoxy)-ethoxy]-benzyl}-1-(6-methyl-3,4-dihydro-2H-quinolin-1-yl)-propan-1-one

Prepared similar to the procedure described in Example 1 but using instead Aldehyde 2 and Amine 6. The title compound was obtained as thick liquid ESI-MS: M⁺: 527.0; HPLC t_{Ret}: 17.72 min

### Example 11

### 3-Amino-2-{4-[2-(2,6-dichloro-4-methyl-phenoxy)-ethoxy]-benzyl}-1-(8-fluoro-3,4-dihydro-2H-quinolin-1-yl)-propan-1-one

Prepared similar to the procedure described in Example 1 but using instead Aldehyde 2 and Amine 7. The title compound was obtained as thick liquid ESI-MS: M⁺: 531.0; HPLC t_{Ret}: 17.12 min

### Example 12

### 3-Amino-2-{4-[2-(2,6-dichloro-4-methyl-phenoxy)-ethoxy]-benzyl}-1-piperidin-1-yl-propan-1-one

Prepared similar to the procedure described in Example 1 but using instead Aldehyde 2 and piperidine. The title compound was obtained as thick liquid ESI-MS: M⁺: 465.1; HPLC t_{Ret}: 16.25 min

### Example 13

### 3-Amino-2-{4-[2-(2,6-dichloro-4-methyl-phenoxy)-ethoxy]-benzyl}-1-(6-methoxy-3,4-dihydro-2H-quinolin-1-yl)-propan-1-one

Prepared similar to the procedure described in Example 1 but using instead Aldehyde 2 and 6-methoxy-1,2,3,4-tetrahydro quinoline. The title compound was obtained as thick liquid ESI-MS: M⁺: 544; HPLC t_{Ret}: 17.09 min

### Example 14

### 3-Amino-1-(6,8-dichloro-3,4-dihydro-2H-quinolin-1-yl)-2-{4-[2-(2,6-dichloro-4-methyl-phenoxy)-ethoxy]-benzyl}-propan-1-one

Prepared similar to the procedure described in Example 1 but using instead Aldehyde 2 and Amine 9. The title compound was obtained as thick liquid ESI-MS: M⁺: 582.8; HPLC t_{Ret}: 18.35 min.

### Example 15

### 3-Amino-2-{4-[2-(2,6-dichloro-4-methyl-phenoxy)-ethoxy]-benzyl}-1-(6-trifluoro methyl-3,4-dihydro-2H -quinolin-1-yl)-propan-1-one

Prepared similar to the procedure described in Example 1 but using instead Aldehyde 2 and Amine 8 The title compound was obtained as thick liquid ESI-MS: M⁺: 580.9; HPLC t_{Ret}: 18.34 min.

### Example 15A

### 3-Amino-2-{4-[2-(2,6-dichloro-4-methyl-phenoxy)-ethoxy]-benzyl}-1-(6-trifluoro methyl-3,4-dihydro-2H -quinolin-1-yl)-propan-1-one hydrochloride

Example 15 (1 eq) was dissolved in 4M dioxane.HCl at 10° C. Mixture was stirred for 20 min at same temperature. Organic volatiles were removed under reduced pressure to get title compound as sticky solid. ESI-MS: M⁺: 581; HPLC t_{Ret}: 18.51 min.

### Example 15B

### 3-Amino-2-{4-[2-(2,6-dichloro-4-methyl-phenoxy)-ethoxy]-benzyl}-1-(6-trifluoro methyl-3,4-dihydro-2H -quinolin-1-yl)-propan-1-one hemifumarate

Example 15 (1 eq) was dissolved in ethanol (5 v) and to this fumaric acid (1 eq) was added and mixture was stirred for 3 h at ambient temperature. Organic volatiles were removed under reduced pressure to afford title compound as white sticky solid. ESI-MS: M⁺: 581; HPLC t_{Ret}: 18.80 min.

### Example 16

### 3-Amino-2-{4-[2-(2,6-dichloro-4-methyl-phenoxy)-ethoxy]-benzyl}-1-(6-methyl-3,4-dihydro-2H-quinolin-1-yl)-propan-1-one

Prepared similar to the procedure described in Example 1 but using instead Aldehyde 2 and Amine 6. The title compound was obtained as thick liquid ESI-MS: M⁺: 527.0; HPLC t_{Ret}: 17.19 min.

### Example 17

### 2-Aminomethyl-3-{4-[2-(2,6-dichloro-4-methyl-phenoxy)-ethoxy]-phenyl}-1-(6,7-dimethoxy-3,4-dihydro-1H-isoquinolin-2-yl)-propan-1-one

Prepared similar to the procedure described in Example 1 but using instead Aldehyde 2 and 6,7-dimethoxy-1,2,3,4-tetrahydro isoquinoline. The title compound was obtained as thick liquid ESI-MS: M⁺: 574.7; HPLC t_{Ret}: 16.24 min.

### Example 18

### 3-Amino-2-{4-[2-(2,6-dichloro-4-methyl-phenoxy)-ethoxy]-benzyl}-1-(4-methyl-piperidin-1-yl)-propan-1-one

Prepared similar to the procedure described in Example 1 but using instead Aldehyde 2 and 4-methyl piperidine. The title compound was obtained as thick liquid ESI-MS: M⁺: 479; HPLC t_{Ret}: 16.85 min.

### Example 19

### 3-Amino-2-{4-[2-(2,6-dichloro-4-methyl-phenoxy)-ethoxy]-benzyl}-1-(4-pyrimidin-2-yl-piperazin-1-yl)-propan-1-one

Prepared similar to the procedure described in Example 1 but using instead Aldehyde 2 and 2-piperazin-1-yl-pyrimidine. The title compound was obtained as thick liquid ESI-MS: M⁺: 544; HPLC t_{Ret}: 15.50 min.

### Example 20

### 3-Amino-2-{4-[2-(2,6-dichloro-4-methyl-phenoxy)-ethoxy]-benzyl}-1-(6-fluoro-3,4-dihydro-2H-quinolin-1-yl)-propan-1-one

Prepared similar to the procedure described in Example 1 but using instead Aldehyde 2 and Amine 1. The title compound was obtained as thick liquid ESI-MS: M⁺: 532.6; HPLC t_{Ret}: 17.45 min.

### Example 21

### 3-Amino-2-{4-[2-(2,6-dichloro-4-methyl-phenoxy)-ethoxy]-benzyl}-1-[4-(2-methoxy-ethoxy)-piperidin-1-yl]-propan-1-one

Prepared similar to the procedure described in Example 1 but using instead Aldehyde 2 and Amine 16. The title compound was obtained as thick liquid ESI-MS: M⁺: 539.1; HPLC t_{Ret}: 15.75 min.

### Example 22

### 2-Aminomethyl-3-{4-[2-(2,6-dichloro-4-methyl-phenoxy)-ethoxy]-phenyl}-1-(4-methyl-piperazin-1-yl)-propan-1-one

Prepared similar to the procedure described in Example 1 but using instead Aldehyde 2 and N-methyl piperazine. The title compound was obtained as thick liquid ESI-MS: M⁺: 480; HPLC t_{Ret}: 13.10 min.

### Example 23

### 3-Amino-2-{4-[2-(2,6-dichloro-4-methyl-phenoxy)-ethoxy]-benzyl}-1-pyrrolidin-1-yl-propan-1-one

Prepared similar to the procedure described in Example 1 but using instead Aldehyde 2 and pyrrolidine. The title compound was obtained as thick liquid ESI-MS: M⁺: 450.9; HPLC t_{Ret}: 15.80 min.

### Example 24

### 3-Amino-2-{4-[2-(2,6-dichloro-4-methyl-phenoxy)-ethoxy]-benzyl}-1-[6-(3-methoxy-propoxy)-3,4-dihydro-2H-quinolin-1-yl]-propan-1-one

Prepared similar to the procedure described in Example 1 but using instead Aldehyde 2 and Amine 13. The title compound was obtained as thick liquid ESI-MS: M⁺: 601; HPLC t_{Ret}: 17.87 min.

### Example 25

### 3-Amino-2-{4-[2-(2,6-dichloro-4-methyl-phenoxy)-ethoxy]-benzyl}-1-morpholin-4-yl-propan-1-one hydrochloride

### Step 1: 2-{4-[2-(2,6-Dichloro-4-methyl-phenoxy)-ethoxy]-benzyl}-3-morpholin-4-yl-3-oxo-propionitrile

Prepared similar to the procedure described in Example 1 but using Aldehyde 2 and morpholine. instead

### Step 2: (2-{4-[2-(2,6-Dichloro-4-methyl-phenoxy)-ethoxy]-benzyl}-3-morpholin-4-yl-3-oxo-propyl)-carbamic acid tert-butyl ester

The compound was prepared following the general process described in Method-B above.

2-{4-[2-(2,6-Dichloro-4-methyl-phenoxy)-ethoxy]-benzyl}-3-morpholin-4-yl-3-oxo-propionitrile (1 eq) obtained from step 1 was dissolved in methanol (15 mL) and to this, added Raney Ni, Boc anhydride (1.2 eq) and triethyl amine (1.5 eq). Reaction mixture was hydrogenate using Parr Apparatus under H₂ atmosphere at 60 psi for 6 hr. Mixture was filtered through hyflow. Hyflow bed was washed with methanol. Organic solvent was evaporated in vacuo to afford title compound as liquid. The crude product was purified by the way of column chromatography (SiO₂, 6:4 (v/v) Hexane:EtOAc) to afford title compound as thick liquid.

### Step 3: 3-Amino-2-{4-[2-(2,6-dichloro-4-methyl-phenoxy)-ethoxy]-benzyl}-1-morpholin-4-yl-propan-1-one hydrochloride

Dioxane.HCl (10%, 2v) was added to (2-{4-[2-(2,6-Dichloro-4-methyl-phenoxy)-ethoxy]-benzyl}-3-morpholin-4-yl-3-oxo-propyl)-carbamic acid tent-butyl ester (1 eq) at 0-5° C. and mixture was stirred for 1 hr at same temperature. Organic volatiles were removed under reduced pressure to afford title compound hydroscopic solid. ESI-MS: M⁺: 466.61; HPLC t_{Ret}: 15.31 min.

The following compounds (Example 26-48) were prepared by following the general process described in Method-B above, at appropriate places as in Example 25.

### Example 26

### 2-Aminomethyl-3-{4-[2-(2,6-dichloro-4-methyl-phenoxy)-ethoxy]-phenyl}-1-(3-methoxymethyl-piperidin-1-yl)-propan-1-one hydrochloride

Prepared similar to the procedure described in Example 25 but using instead Aldehyde 2 and Amine 14. The title compound was obtained as hygroscopic solid ESI-MS: M⁺: 508.6; HPLC t_{Ret}: 16.60 min.

### Example 27

### 3-Amino-2-{4-[2-(2,6-dichloro-4-methyl-phenoxy)-ethoxy]-benzyl}-1-(6-trifluoromethoxy-3,4-dihydro-2H-quinolin-1-yl)-propan-1-one hydrochloride

Prepared similar to the procedure described in Example 25 but using instead Aldehyde 2 and Amine 10. The title compound was obtained as hygroscopic solid ESI-MS: M⁺: 596.7; HPLC t_{Ret}: 18.75 min.

### Example 28

### 3-Amino-2-{4-[2-(2,6-dichloro-4-methyl-phenoxy)-ethoxy]-benzyl}-1-[6-(2,2,2-trifluoro-ethoxy)-3,4-dihydro-2H-quinolin-1-yl]-propan-1-one hydrochloride

Prepared similar to the procedure described in Example 25 but using Aldehyde 2 and Amine 12 instead. The title compound was obtained as hygroscopic solid ESI-MS: M⁺: 611; HPLC t_{Ret}: 18.56 min.

### Example 29

### D-(2-Aminomethyl-3-{4-[2-(2,6-dichloro-4-methyl-phenoxy)-ethoxy]-phenyl}-propionyl)-pyrrolidine-2-carboxylic acid methyl ester hydrochloride

Prepared similar to the procedure described in Example 25 but using Aldehyde 2 and D-proline methyl ester instead. The title compound was obtained as mixture of diastereomers and nature was hygroscopic solid ESI-MS: m⁺: 509; HPLC t_{Ret}: 16.32 & 16.47 min.

### Example 30

### 2-Aminomethyl-3-{4-[2-(2,6-dichloro-4-methyl-phenoxy)-ethoxy]-phenyl}-1-(3-methoxy-piperidin-1-yl)-propan-1-one hydrochloride

Prepared similar to the procedure described in Example 25 but using Aldehyde 2 and Amine 15 instead. The title compound was obtained as hygroscopic solid ESI-MS: 495.7; HPLC t_{Ret}: 16.22 min.

### Example 31

### 3-Amino-2-{4-[2-(2,6-dichloro-phenoxy)-ethoxy]-benzyl}-1-(6-trifluoromethyl-3,4-dihydro-2H-quinolin-1-yl)-propan-1-one hydrochloride

Prepared similar to the procedure described in Example 25 but using Aldehyde 3 and Amine 8 instead. The title compound was obtained as hygroscopic solid ESI-MS: M⁺: 567; HPLC t_{Ret}: 17.77 min.

### Example 31A

3-Amino-2-{4-[2-(2,6-dichloro-phenoxy)-ethoxy]-benzyl}-1-(6-trifluoromethyl-3,4-dihydro-2H-quinolin-1-yl)-propan-1-one hydrochloride synthesized as per the procedure reported in Example 31 was subjected to purification by HPLC using a chiral stationary phase Regis (R, R) Whelk-01 column, 10/100 FEC [250*4.6 mm], 5 u, 1.5 ml/min, mobile phase (90:10) N-hexane:0.1% TEA in Ethanol over 40 min yielded non-polar isomer ESI-MS: M⁺ 566.93, HPLC Chiral Regis Whelk column t_{Ret} 32.32 min.

### Example 31B

3-Amino-2-{4-[2-(2,6-dichloro-phenoxy)-ethoxy]-benzyl}-1-(6-trifluoromethyl-3,4-dihydro-2H-quinolin-1-yl)-propan-1-one hydrochloride synthesized as per the procedure reported in Example 31 was subjected to purification by HPLC using a chiral stationary phase Regis (R, R) Whelk-01 column, 10/100 FEC [250*4.6 mm], 5 u, 1.5 ml/min, mobile phase (90:10) N-hexane:0.1% TEA in Ethanol over 40 min yielded polar isomer ESI-MS: 566.90, HPLC Chiral Regis Whelk column t_{Ret} 20.40 min.

### Example 32

### 3-Amino-2-{4-[2-(2,6-dichloro-phenoxy)-ethoxy]-benzyl}-1-(6-trifluoromethoxy-3,4-dihydro-2H-quinolin-1-yl)-propan-1-one hydrochloride

Prepared similar to the procedure described in Example 25 but using Aldehyde 3 and Amine 10 instead. The title compound was obtained as hygroscopic solid ESI-MS: M⁺: 582.6; HPLC t_{Ret}: 18.12 min.

### Example 33

### 3-Amino-2-{4-[2-(2-chloro-4-methyl-phenoxy)-ethoxy]-benzyl}-1-(6-trifluoromethyl-3,4-dihydro-2H-quinolin-1-yl)-propan-1-one

Prepared similar to the procedure described in Example 25 but using Aldehyde 12 and Amine 8 instead. The title compound was obtained as hygroscopic solid ESI-MS: M⁺: 547; HPLC t_{Ret}: 17.97 min.

### Example 34

### 3-Amino-2-{4-[2-(2,6-difluoro-phenoxy)-ethoxy]-benzyl}-1-(6-trifluoromethyl-3,4-dihydro-2H-quinolin-1-yl)-propan-1-one hydrochloride

Prepared similar to the procedure described in Example 25 but using Aldehyde 4 and Amine 8 instead. The title compound was obtained as hygroscopic solid ESI-MS: M⁺: 534.6; HPLC t_{Ret}: 17.05 min.

### Example 35

### 3-Amino-2-{4-[2-(2,6-difluoro-phenoxy)-ethoxy]-benzyl}-1-(6-fluoro-3,4-dihydro-2H-quinolin-1-yl)-propan-1-one hydrochloride

Prepared similar to the procedure described in Example 25 but using Aldehyde 4 and Amine 1 instead. The title compound was obtained as hygroscopic solid ESI-MS: 484.7; HPLC t_{Ret}: 16.09 min.

### Example 36

### 3-Amino-2-{4-[2-(2,4-difluoro-phenoxy)-ethoxy]-benzyl}-1-(6-trifluoromethyl-3,4-dihydro-2H-quinolin-1-yl)-propan-1-one hydrochloride

Prepared similar to the procedure described in Example 25 but using Aldehyde 5 and Amine 8 instead. The title compound was obtained as hygroscopic solid ESI-MS: M⁺: 534.7; HPLC t_{Ret}: 17.08 min.

### Example 37

### 3-Amino-2-{4-[2-(2,4-dichloro-phenoxy)-ethoxy]-benzyl}-1-(6-fluoro-3,4-dihydro-2H-quinolin-1-yl)-propan-1-one hydrochloride

Prepared similar to the procedure described in Example 25 but using Aldehyde 6 and Amine 1 instead. The title compound was obtained as hygroscopic solid ESI-MS: M⁺: 517; HPLC t_{Ret}: 17.42 min.

### Example 38

### 3-Amino-2-{4-[2-(2,4-dichloro-phenoxy)-ethoxy]-benzyl}-1-(6-trifluoromethyl-3,4-dihydro-2H-quinolin-1-yl)-propan-1-one hydrochloride chloride

Prepared similar to the procedure described in Example 25 but using Aldehyde 6 and Amine 8 instead. The title compound was obtained as hygroscopic solid ESI-MS: M⁺: 567; HPLC t_{Ret}: 18.37 min.

### Example 39

### 3-Amino-2-{4-[2-(4-chloro-2-fluoro-phenoxy)-ethoxy]-benzyl}-1-(6-fluoro-3,4-dihydro-2H-quinolin-1-yl)-propan-1-one hydrochloride

Prepared similar to the procedure described in Example 25 but using Aldehyde 7 and Amine 1 instead. The title compound was obtained as hygroscopic solid ESI-MS: M⁺: 501; HPLC t_{Ret}: 16.48 min.

### Example 40

### 3-Amino-2-{4-[2-(4-chloro-2-fluoro-phenoxy)-ethoxy]-benzyl}-1-(6-trifluoromethyl-3,4-dihydro-2H-quinolin-1-yl)-propan-1-one hydrochloride

Prepared similar to the procedure described in Example 25 but using Aldehyde 7 and Amine 8 instead. The title compound was obtained as hygroscopic solid ESI-MS: M⁺: 551.3; HPLC t_{Ret}: 18.74 min.

### Example 41

### 3-Amino-2-{6-[2-(2,6-dichloro-4-methyl-phenoxy)-ethoxy]-pyridin-3-ylmethyl}-1-(6-trifluoromethyl-3,4-dihydro-2H-quinolin-1-yl)-propan-1-one

Prepared similar to the procedure described in Example 1 but using Aldehyde 8 and Amine 8 instead. The title compound was obtained as thick liquid ESI-MS: M⁺: 582; HPLC t_{Ret}: 17.84 min

### Example 42

### 2-Aminomethyl-3-{6-[2-(2,6-dichloro-4-methyl-phenoxy)-ethoxy]-pyridin-3-yl}-1-piperidin-1-yl-propan-1-one

Prepared similar to the procedure described in Example 1 but using Aldehyde 8 and piperidine instead. The title compound was obtained as thick liquid ESI-MS: M⁺: 466; HPLC t_{Ret}: 15.53 min.

### Example 43

### 2-Aminomethyl-3-{6-[2-(2,6-dichloro-4-methyl-phenoxy)-ethoxy]-pyridin-3-yl}-1-(3,4-dihydro-2H-quinolin-1-yl)-propan-1-one

Prepared similar to the procedure described in Example 1 but using Aldehyde 8 and 1,2,3,4-tetrahydro quinoline instead. The title compound was obtained as thick liquid ESI-MS: M⁺: 514; HPLC t_{Ret}: 16.69 min.

### Example 44

### 3-Amino-2-{6-[2-(2,6-dichloro-4-methyl-phenoxy)-ethoxy]-pyridin-3-ylmethyl}-1-(6-fluoro-3,4-dihydro-2H-quinolin-1-yl)-propan-1-one hydrochloride

Prepared similar to the procedure described in Example 25 but using Aldehyde 8 and Amine 1 instead. The title compound was obtained as hygroscopic solid ESI-MS: M⁺: 533.3 ; HPLC t_{Ret}: 16.79 min.

### Example 45

### 2-Aminomethyl-3-{4-[3-(2,6-dichloro-4-methyl-phenoxy)-propoxy]-phenyl}-1-(3,4-dihydro-2H-quinolin-1-yl)-propan-1-one

Prepared similar to the procedure described in Example 1 but using Aldehyde 9 and 1,2,3,4-tetrahydro quinoline instead. The title compound was obtained as thick liquid ESI-MS: M⁺: 527; HPLC t_{Ret}: 18.10 min.

### Example 46

### 3-Amino-1-(6-fluoro-3,4-dihydro-2H-quinolin-1-yl)-2-{4-[3-(2-methoxy-benzyloxy)-propoxy]-benzyl}-propan-1-one

Prepared similar to the procedure described in Example 1 but using Aldehyde 10 and Amine 1 instead. The title compound was obtained as thick liquid ESI-MS: M⁺: 507 ; HPLC t_{Ret}: 16.55 min.

### Example 47

### 3-Amino-2-{4-[3-(2-methoxy-benzyloxy)-propoxy]-benzyl}-1-(6-trifluoromethyl-3,4-dihydro-2H-quinolin-1-yl)-propan-1-one

Prepared similar to the procedure described in Example 1 but using Aldehyde 10 and Amine 8 instead. The title compound was obtained as thick liquid ESI-MS: M⁺: 557; HPLC t_{Ret}: 17.54 min.

### Example 48

### 3-Amino-2-{4-[2-(2,6-dichloro-4-methyl-phenoxy)-ethoxy]-3-methoxy-benzyl}-1-(3,4-dihydro-2H-quinolin-1-yl)-propan-1-one hydrochloride

Prepared similar to the procedure described in Example 25 but using Aldehyde 11 and 1,2,3,4-tetrahydroquinoline instead. The title compound was obtained as hygroscopic solid ESI-MS: M⁺: 543; HPLC t_{Ret}: 16.93 min.

### Example 49

### 1-(3-(6-Chloro-3,4-dihydro-2H-quinolin-1-yl)-2-{4-[2-(2,6-dichloro-4-methyl-phenoxy)-ethoxy]-benzyl}-3-oxo-propyl)-3-phenyl-urea

### Step-1: 3-Amino-1-(6-chloro-3,4-dihydro-2H-quinolin-1-yl)-2-{4-[2-(2,6-dichloro-4-methyl-phenoxy)-ethoxy]-benzyl}-propan-1-one

Prepared similar to the procedure described in Example 5.

### Step-2

3-Amino-1-(6-chloro-3,4-dihydro-2H-quinolin-1-yl)-2-{4-[2-(2,6-dichloro-4-methyl-phenoxy)-ethoxy]-benzyl}-propan-1-one (1eq), phenyl isocyanate (1eq) and triethyl amine 1.2 eq) were taken in dichloromethane. Mixture was stirred for 6 h at room temperature. Mixture was quenched in water; product was extracted with EtOAc. Organic layer was washed with brine, dried over sodium sulfate, filtered and evaporated in vacuo to afford brown sticky solid. ESI-MS. (M+23) 690, HPLC t_{Ret} : 25.80 min.

### Example 50

### [2-{4-[2-(2,6-Dichloro-4-methyl-phenoxy)-ethoxy]-benzyl}-3-oxo-3-(6-trifluoromethyl-3,4-dihydro-2H -quinolin-1-yl)-propyl]-carbamic acid tent-butyl ester

### Step 1: 3-Amino-2-{4-[2-(2,6-dichloro-4-methyl-phenoxy)-ethoxy]-benzyl}-1-(6-trifluoro methyl-3,4-dihydro-2H -quinolin-1-yl)-propan-1-one was prepared similar to the procedure described in Example 15

### Step 2

3-Amino-2-{4-[2-(2,6-dichloro-4-methyl-phenoxy)-ethoxy]-benzyl}-1-(6-trifluoromethyl-3,4-dihydro-2H -quinolin-1-yl)-propan-1-one (1 eq) obtained from step 1, Boc anhydride (1.1eq), triethylamine (1.3 eq) were mixed in DCM (5 v) and stirred for 3 h at 25° C. Mixture was diluted with water. Product was extracted with EtOAc. Organic layer was washed with brine, dried over sodium sulfate, filtered, and evaporated in vacuo to afford thick liquid. ESI-MS. (M+23) 704.7 HPLC t_{Ret}: 27.09 min.

### Example 51

### 3-Amino-2-{4-[2-(2,6-dichloro-phenyl)-ethoxy]-benzyl}-1-(6-trifluoromethyl-3,4-dihydro-2H-quinolin-1-yl)-propan-1-one hydrochloride

Prepared similar to the procedure described in Example 25 but using Aldehyde 13 and Amine 8 instead. The title compound was obtained as hygroscopic solid ESI-MS: M⁺: 551.4; HPLC t_{Ret}: 13.44 min.

### Example 52

### 3-Amino-2-{4-[2-(2,6-dichloro-phenyl)-ethoxy]-benzyl}-1-(6-fluoro-3,4-dihydro-2H-quinolin-1-yl)-propan-1-one hydrochloride

Prepared similar to the procedure described in Example 25 but using Aldehyde 13 and Amine 1 instead. The title compound was obtained as hygroscopic solid ESI-MS: M⁺: 500.7; HPLC t_{Ret}: 11.99 min.

### Example 53

### 3-Amino-1-(6-tert-butyl-3,4-dihydro-2H-quinolin-1-yl)-2-{4-[2-(2,6-dichloro-phenoxy)-ethoxy]-benzyl}-propan-1-one hydrochloride

Prepared similar to the procedure described in Example 25 but using Aldehyde 3 and Amine 17 instead. The title compound was obtained as hygroscopic solid ESI-MS: (M+H)⁺: 556.82; HPLC t_{Ret}: 18.86 min.

### Example 54

### 3-Amino-2-{4-[2-(piperidin-4-yloxy)-ethoxy]-benzyl}-1-(6-trifluoromethyl-3,4-dihydro-2H-quinolin-1-yl)-propan-1-one hydrochloride

Prepared similar to the procedure described in Example 25 but using Aldehyde 14 and Amine 8 instead. The title compound was obtained as hygroscopic solid ESI-MS: (M)⁺: 505.6; HPLC t_{Ret}: 12.03 min.

### Example 55

### 3-Amino-1-(3-amino-pyrrolidin-1-yl)-2-{4-[2-(2,6-dichloro-phenoxy)-ethoxy]-benzyl}-propan-1-one

Prepared similar to the procedure described in Example 1 but using Aldehyde 3 and 3-amino-pyrrolidine-1-carboxylic acid tert-butyl ester, instead. The title compound was obtained as hygroscopic solid ESI-MS: (M)⁺: 551.7; HPLC t_{Ret}: 16.098 min.

### Example 56

### 2-Aminomethyl-3-{4-[2-(2,6-dichloro-phenoxy)-ethoxy]-phenyl}-1-(2,3-dihydro-indol-1-yl)-propan-1-one hydrochloride

Prepared similar to the procedure described in Example 25 but using Aldehyde 3 and 2,3-dihydro-1H-indole, instead. The title compound was obtained as hygroscopic solid ESI-MS: (M+H)⁺: 487.7 HPLC t_{Ret}: 16.75 min.

### Example 57

### 3-Amino-2-{4-[2-(2,4-difluoro-phenoxy)-ethoxy]-benzyl}-1-(6-trifluoromethoxy-3,4-dihydro-2H-quinolin-1-yl)-propan-1-one hydrochloride

Prepared similar to the procedure described in Example 25 but using Aldehyde 5 and Amine 12, instead. The title compound was obtained as hygroscopic solid ESI-MS: (M+H)⁺: 550.7; HPLC t_{Ret}: 17.273 min.

### Example 58

### 3-Amino-2-{4-[2-(2,4-difluoro-phenoxy)-ethoxy]-benzyl}-1-piperidin-1-yl-propan-1-one hydrochloride

Prepared similar to the procedure described in Example 25 but using Aldehyde 5 and piperidine instead. The title compound was obtained as hygroscopic solid ESI-MS: (M)⁺: 418.90; HPLC t_{Ret}: 15.036 min.

### Example 59

### 3-Amino-2-{4-[2-(2,4-difluoro-phenoxy)-ethoxy]-benzyl}-1-(3,4-dihydro-2H-quinolin-1-yl)-propan-1-one hydrochloride

Prepared similar to the procedure described in Example 25 but using Aldehyde 5 and 1,2,3,4-tetrahydroquinoline, instead. The title compound was obtained as hygroscopic solid ESI-MS: (M)⁺: 467; HPLC t_{Ret}: 16.011 min.

Biological Data:

In-Vitro Renin Inhibition Assay

The enzymatic in vitro assay was performed in 96 well polypropylene plate (Nunc), using a modified Renin inhibitor screening assay protocol (Cayman, cat no: 10006270). The reaction system comprised assay buffer containing 50 mM Tris-HCL, pH=8.0 & 100 mM sodium chloride, human recombinant renin (1:20 diluted with fixed activity), synthetic renin substrates (9.5 μM) and different concentrations of renin inhibitors in DMSO in a total reaction system of 100 μl. The entire incubation mixture were incubated at 37° C. for 30 mins and the fluorescence was read in kinetic mode using excitation wavelengths of 335-345 nm and emission wavelengths of 485-510 nm. Enzyme inhibition was determined by percent inhibition of renin activity.

The following table shows the Renin inhibition of selected compounds at 1μM and 0.1 μM concentration.

| Compounds | 1 μM | 0.1 μM |
|---|---|---|
| Example 2 | 97.3 | 90.5 |
| Example 4 | 73.5 | — |
| Example 5 | 79.62 | — |
| Example 6 | 86.48 | — |
| Example 9 | 98.13 | 80.49 |
| Example 11 | 68.53 | — |
| Example 12 | 90.71 | 77.0 |
| Example 13 | 96.53 | 32.1 |
| Example 14 | 86.96 | 41.79 |
| Example 15 | 98.66 | 99.19 |
| Example 15A | 88.0 | 80.00 |
| Example 16 | 87.41 | 52.65 |
| Example 17 | 53.57 | — |
| Example 18 | 65.83 | — |
| Example 20 | 96.11 | 67.96 |
| Example 23 | 81.10 | 59.53 |
| Example 24 | 93.19 | 90.71 |
| Example 25 | 76.40 | 45.08 |
| Example 26 | 93.93 | 92.80 |
| Example 27 | 99.18 | 100.0 |
| Example 28 | — | 73.76 |
| Example 29 | — | 90.3 |
| Example 30 | 92.93 | 92.80 |
| Example 31 | 102.36 | 94.13 |
| Example 32 | — | 100.0 |
| Example 33 | 89.22 | 92.88 |
| Example 34 | — | 31.23 |
| Example 35 | — | 26.27 |
| Example 36 | — | 45.04 |
| Example 37 | — | 36.38 |
| Example 38 | — | 35.41 |
| Example 39 | — | 36.30 |
| Example 40 | — | 38.93 |
| Example 41 | 98.60 | 80.10 |
| Example 42 | 82.45 | 71.05 |
| Example 43 | 64.24 | — |
| Example 44 | 96.27 | 63.10 |
| Example 45 | 97.06 | 18.7 |
| Example 47 | — | 43.08 |
| Example 48 | 58.47 | — |
| Example 53 | — | 93.08 |

Following table represents measured IC₅₀ values of the selected compounds for its Renin inhibition in human plasma.

| Compounds | IC₅₀ nM |
|---|---|
| Example 12 | 70.15 |
| Example 15A | 7.85 |
| Example 26 | 27.75 |
| Example 27 | 0.921 |
| Example 31 | 28.69 |
| Example 32 | 0.829 |
| Example 24 | 25.41 |

The compounds of the present invention were found to be inhibitors of renin and found to be safe and non-toxic.

1. Compound represented by the formula (1) 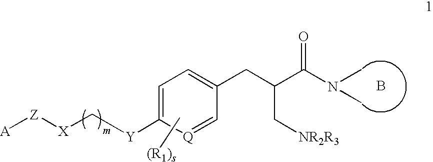
wherein
m is an integer selected from 1 to 4; Z represents either a bond or —CH₂—; X and Y are each independently selected from the group comprising of —CH₂—, O, and S(O)p; p in each instance in which it occur is independently 0, 1 or 2;
A is an optionally substituted aryl or a 5-6 membered heterocyclic ring containing 1 to 3 heteroatoms selected from O, S, and N;
Q represents a carbon or nitrogen atom;
B represents a saturated, unsaturated or partly unsaturated single or fused heterocyclic ring which optionally contains one or more additional heteroatoms selected from nitrogen, oxygen and sulphur or may comprise an —SO— or an —SO₂— group; wherein the heterocyclic ring as defined above comprises one or more nitrogen atom, such nitrogen atoms are optionally be substituted with optionally substituted groups selected from C₁-C₈-alkyl, C₁-C₈-alkanoyl, aryl and heterocyclic group wherein when B represents a fused ring then the ring system preferably contains 9-16 member heterocyclic groups; R₁ at each occurrence independently represents hydrogen, halogen, cyano, optionally substituted C₁-C₆ alkyl, or optionally substituted C₁-C₆ alkoxy groups; s represents an integer selected from 0 to 3; R₂, and R₃ may be same or different and are independently selected from the group comprising of hydrogen, optionally substituted C₁-C₆ alkyl, C₁-C₆ alkoxy, C(O)Rb, and C(O)NRcRd groups;
Rb is selected from the group comprising of hydrogen, optionally substituted C₁-C₆ alkyl, and optionally substituted C₁-C₆ alkoxy groups; and Rc and Rd are independently selected from the groups comprising hydrogen, optionally substituted C₁-C₆ alkyl, and unsubstituted or substituted aryl groups.

2. The compound as claimed in claim 1 wherein the substituents on A are selected from one, two, three or four substituents independently representing OH, CN, halogen, N₃, NO₂, COOH, OCHF₂H, CF₃, C(₁₋₆) alkyl, C(₂₋₆) alkenyl, C(₁₋₆) alkoxy, C(O)C₁-C₆ alkyl, S(O)p C(₁₋₆) alkyl, and (CH₂)1-2Oalkyl groups.

3. The compound as claimed in claim 1 wherein the substituents on heterocycle B are selected from halogen, hydroxyl, oxide, oxo, cyano, and optionally substituted groups selected from haloalkyl, haloalkoxy, C₁-C₈-alkyl, C₁-C₈-alkoxy, C₁-C₈-alkoxy-C₁-C₈-alkyl, C₁C₈alkoxy-C₁C₈alkoxy, C₁-C₈-alkoxycarbonylamino, C₁-C₈-alkylcarbonylamino, C₁-C₈-alkylamino, N,N-di-C₁-C₈-alkylamino, aryl-C₀-C₄-alkyl, aryloxy-C₀-C₄-alkyl, aryl-C₀-C₄-alkyl-C₁-C₈-alkoxy, aryloxy-C₈-C₄-alkyl-C₁-C₈-alkoxy, heterocyclyl-C₀-C₄-alkyl, heterocyclyloxy-C₀-C₄-alkyl, heterocyclyl-C₀-C₄-alkyl-C₁-C₈-alkoxy and heterocyclyloxy-C₀-C₄-alkyl-C₁-C₈-alkoxy groups.

4. The compound as claimed in claim 1 wherein the group representing A is selected from optionally substituted phenyl, benzyl, and piperidinyl groups.

5. The compound as claimed in claim 1 wherein the group B forms an optionally substituted 1,2,3,4-tetrahydroquinoline, 1,2,3,4-tetrahydroisoquinoline, piperidine, morpholine, pyrrolidine, piperazine, indoline and or indole ring or their suitable derivatives.

6. The compound as claimed in claim 1 selected from 3-Amino-1-(3,4-dihydro-2H-quinolin-1-yl)-2-{4-[2-(2,5-dimethyl-phenoxy)-ethoxy]-benzyl}-propan-1-one;
3-Amino-2-{4-[2-(2,6-dichloro-4-methyl-phenoxy)-ethoxy]-benzyl}-1-(3,4-dihydro-2H-quinolin-1-yl)-propan-1-one;
3-Amino-2-{4-[2-(2,6-dichloro-4-methyl-phenoxy)-ethoxy]-benzyl}-1-(3,4-dihydro-1H-isoquinolin-2-yl)-propan-1-one;
3-Amino-2-{4-[2-(2,6-dichloro-4-methyl-phenoxy)-ethoxy]-benzyl}-1-(2,3-dihydro-benzo[1,4]oxazin-4-yl)-propan-1-one;
3-Amino-1-(6-chloro-3,4-dihydro-2H-quinolin-1-yl)-2-{4-[2-(2,6-dichloro-4-methyl-phenoxy)-ethoxy]-benzyl}-propan-1-one;
3-Amino-1-(6-bromo-3,4-dihydro-2H-quinolin-1-yl)-2-{4-[2-(2,6-dichloro-4-methyl-phenoxy)-ethoxy]-benzyl}-propan-1-one;
3-Amino-1-(7,8-dichloro-3,4-dihydro-2H-quinolin-1-yl)-2-{4-[2-(2,6-dichloro-4-methyl-phenoxy)-ethoxy]-benzyl}-propan-1-one;
3-Amino-2-{4-[2-(2,6-dichloro-4-methyl-phenoxy)-ethoxy]-benzyl}-1-(8-methoxy-3,4-dihydro-2H-quinolin-1-yl)-propan-1-one;
3-Amino-2-{4-[2-(2,6-dichloro-4-methyl-phenoxy)-ethoxy]-benzyl}-1-(7,8-dimethyl-3,4-dihydro-2H-quinolin-1-yl)-propan-1-one;
3-Amino-2-{4-[2-(2,6-dichloro-4-methyl-phenoxy)-ethoxy]-benzyl}-1-(6-methyl-3,4-dihydro-2H-quinolin-1-yl)-propan-1-one;
3-Amino-2-{4-[2-(2,6-dichloro-4-methyl-phenoxy)-ethoxy]-benzyl}-1-(8-fluoro-3,4-dihydro-2H-quinolin-1-yl)-propan-1-one;
3-Amino-2-{4-[2-(2,6-dichloro-4-methyl-phenoxy)-ethoxy]-benzyl}-1-piperidin-1-yl-propan-1-one;
3-Amino-2-{4-[2-(2,6-dichloro-4-methyl-phenoxy)-ethoxy]-benzyl}-1-(6-methoxy-3,4-dihydro-2H-quinolin-1-yl)-propan-1-one;
3-Amino-1-(6,8-dichloro-3,4-dihydro-2H-quinolin-1-yl)-2-{4-[2-(2,6-dichloro-4-methyl-phenoxy)-ethoxy]-benzyl}-propan-1-one;
3-Amino-2-{4-[2-(2,6-dichloro-4-methyl-phenoxy)-ethoxy]-benzyl}-1-(6-trifluoromethyl-3,4-dihydro-2H -quinolin-1-yl)-propan-1-one;
3-Amino-2-{4-[2-(2,6-dichloro-4-methyl-phenoxy)-ethoxy]-benzyl}-1-(6-trifluoromethyl-3,4-dihydro-2H -quinolin-1-yl)-propan-1-one hydrochloride salt;
3-Amino-2-{4-[2-(2,6-dichloro-4-methyl-phenoxy)-ethoxy]-benzyl}-1-(6-trifluoromethyl-3,4-dihydro-2H-quinolin-1-yl)-propan-1-one hemifumarate;
3-Amino-2-{4-[2-(2,6-dichloro-4-methyl-phenoxy)-ethoxy]-benzyl}-1-(6-methyl-3,4-dihydro-2H-quinolin-1-yl)-propan-1-one;
2-Aminomethyl-3-{4-[2-(2,6-dichloro-4-methyl-phenoxy)-ethoxy]-phenyl}-1-(6,7-dimethoxy-3,4-dihydro-1H-isoquinolin-2-yl)-propan-1-one;
3-Amino-2-{4-[2-(2,6-dichloro-4-methyl-phenoxy)-ethoxy]-benzyl}-1-(4-methyl-piperidin-1-yl)-propan-1-one;
3-Amino-2-{4-[2-(2,6-dichloro-4-methyl-phenoxy)-ethoxy]-benzyl}-1-(4-pyrimidin-2-yl-piperazin-1-yl)-propan-1-one;
3-Amino-2-{4-[2-(2,6-dichloro-4-methyl-phenoxy)-ethoxy]-benzyl}-1-(6-fluoro-3,4-dihydro-2H-quinolin-1-yl)-propan-1-one;
3-Amino-2-{4-[2-(2,6-dichloro-4-methyl-phenoxy)-ethoxy]-benzyl}-1-[4-(2-methoxy-ethoxy)-piperidin-1-yl]-propan-1-one;
2-Aminomethyl-3-{4-[2-(2,6-dichloro-4-methyl-phenoxy)-ethoxy]-phenyl}-1-(4-methyl-piperazin-1-yl)-propan-1-one;
3-Amino-2-{4-[2-(2,6-dichloro-4-methyl-phenoxy)-ethoxy]-benzyl}-1-pyrrolidin-1-yl-propan-1-one;
3-Amino-2-{4-[2-(2,6-dichloro-4-methyl-phenoxy)-ethoxy]-benzyl}-1-[6-(3-methoxy-propoxy)-3,4-dihydro-2H-quinolin-1-yl]-propan-1-one;
3-Amino-2-{4-[2-(2,6-dichloro-4-methyl-phenoxy)-ethoxy]-benzyl}-1-morpholin-4-yl-propan-1-one hydrochloride;
2-Aminomethyl-3-{4-[2-(2,6-dichloro-4-methyl-phenoxy)-ethoxy]-phenyl}-1-(3-methoxymethyl-piperidin-1-yl)-propan-1-one hydrochloride;
3-Amino-2-{4-[2-(2,6-dichloro-4-methyl-phenoxy)-ethoxy]-benzyl}-1-(6-trifluoromethoxy-3,4-dihydro-2H-quinolin-1-yl)-propan-1-one hydrochloride;
3-Amino-2-{4-[2-(2,6-dichloro-4-methyl-phenoxy)-ethoxy]-benzyl}-1-[6-(2,2,2-trifluoro-ethoxy)-3,4-dihydro-2H-quinolin-1-yl]-propan-1-one hydrochloride;
1-(2-Aminomethyl-3-{4-[2-(2,6-dichloro-4-methyl-phenoxy)-ethoxy]-phenyl}-propionyl)-pyrrolidine-2-carboxylic acid methyl ester hydrochloride;
2-Aminomethyl-3-{4-[2-(2,6-dichloro-4-methyl-phenoxy)-ethoxy]-phenyl}-1-(3-methoxy-piperidin-1-yl)-propan-1-one hydrochloride;
3-Amino-2-{4-[2-(2,6-dichloro-phenoxy)-ethoxy]-benzyl}-1-(6-trifluoromethyl-3,4-dihydro-2H-quinolin-1-yl)-propan-1-one hydrochloride;
3-Amino-2-{4-[2-(2,6-dichloro-phenoxy)-ethoxy]-benzyl}-1-(6-trifluoromethoxy-3,4-dihydro-2H-quinolin-1-yl)-propan-1-one hydrochloride;
3-Amino-2-{4-[2-(2-chloro-4-methyl-phenoxy)-ethoxy]-benzyl}-1-(6-trifluoromethyl-3,4-dihydro-2H-quinolin-1-yl)-propan-1-one;
3-Amino-2-{4-[2-(2,6-difluoro-phenoxy)-ethoxy]-benzyl}-1-(6-trifluoromethyl-3,4-dihydro-2H-quinolin-1-yl)-propan-1-one hydrochloride;
3-Amino-2-{4-[2-(2,6-difluoro-phenoxy)-ethoxy]-benzyl}-1-(6-fluoro-3,4-dihydro-2H-quinolin-1-yl)-propan-1-one hydrochloride;
3-Amino-2-{4-[2-(2,4-difluoro-phenoxy)-ethoxy]-benzyl}-1-(6-trifluoromethyl-3,4-dihydro-2H-quinolin-1-yl)-propan-1-one hydrochloride;
3-Amino-2-{4-[2-(2,4-dichloro-phenoxy)-ethoxy]-benzyl}-1-(6-fluoro-3,4-dihydro-2H-quinolin-1-yl)-propan-1-one hydrochloride;
3-Amino-2-{4-[2-(2,4-dichloro-phenoxy)-ethoxy]-benzyl}-1-(6-trifluoromethyl-3,4-dihydro-2H-quinolin-1-yl)-propan-1-one hydrochloride chloride;
3-Amino-2-{4-[2-(4-chloro-2-fluoro-phenoxy)-ethoxy]-benzyl}-1-(6-fluoro-3,4-dihydro-2H-quinolin-1-yl)-propan-1-one hydrochloride;
3-Amino-2-{4-[2-(4-chloro-2-fluoro-phenoxy)-ethoxy]-benzyl}-1-(6-trifluoromethyl-3,4-dihydro-2H-quinolin-1-yl)-propan-1-one hydrochloride;
3-Amino-2-{6-[2-(2,6-dichloro-4-methyl-phenoxy)-ethoxy]-pyridin-3-ylmethyl}-1-(6-trifluoromethyl-3,4-dihydro-2H-quinolin-1-yl)-propan-1-one;
2-Aminomethyl-3-{6-[2-(2,6-dichloro-4-methyl-phenoxy)-ethoxy]-pyridin-3-yl}-1-piperidin-1-yl-propan-1-one;
2-Aminomethyl-3-{6-[2-(2,6-dichloro-4-methyl-phenoxy)-ethoxy]-pyridin-3-yl}-1-(3,4-dihydro-2H-quinolin-1-yl)-propan-1-one;
3-Amino-2-{6-[2-(2,6-dichloro-4-methyl-phenoxy)-ethoxy]-pyridin-3-ylmethyl}-1-(6-fluoro-3,4-dihydro-2H-quinolin-1-yl)-propan-1-one hydrochloride;
2-Aminomethyl-3-{4-[3-(2,6-dichloro-4-methyl-phenoxy)-propoxy]-phenyl}-1-(3,4-dihydro-2H-quinolin-1-yl)-propan-1-one;
3-Amino-1-(6-fluoro-3,4-dihydro-2H-quinolin-1-yl)-2-{4-[3-(2-methoxy-benzyloxy)-propoxy]-benzyl}-propan-1-one;
3-Amino-2-{4-[3-(2-methoxy-benzyloxy)-propoxy]-benzyl}-1-(6-trifluoromethyl-3,4-dihydro-2H-quinolin-1-yl)-propan-1-one;
3-Amino-2-{4-[2-(2,6-dichloro-4-methyl-phenoxy)-ethoxy]-3-methoxy-benzyl}-1-(3,4-dihydro-2H-quinolin-1-yl)-propan-1-one hydrochloride;
1-(3-(6-Chloro-3,4-dihydro-2H-quinolin-1-yl)-2-{4-[2-(2,6-dichloro-4-methyl-phenoxy)-ethoxy]-benzyl}-3-oxo-propyl)-3-phenyl-urea;
[2-{4-[2-(2,6-Dichloro-4-methyl-phenoxy)-ethoxy]-benzyl}-3-oxo-3-(6-trifluoromethyl-3,4-dihydro-2H-quinolin-1.-yl)-propyl]-carbamic acid tert-butyl ester;
3-Amino-2-{4-[2-(2,6-dichloro-phenyl)-ethoxy]-benzyl}-1-(6-trifluoromethyl-3,4-dihydro-2H-quinolin-1-yl)-propan-1-one hydrochloride;
3-Amino-2-{4-[2-(2,6-dichloro-phenyl)-ethoxy]-benzyl}-1-(6-fluoro-3,4-dihydro-2H-quinolin-1-yl)-propan-1-one hydrochloride;
3-Amino-1-(6-tert-butyl-3,4-dihydro-2H-quinolin-1-yl)-2-{4-[2-(2,6-dichloro-phenoxy)-ethoxy]-benzyl}-propan-1-one hydrochloride;
3-Amino-2-{4-[2-(piperidin-4-yloxy)-ethoxy]-benzyl}-1-(6-trifluoromethyl-3,4-dihydro-2H-quinolin-1-yl)-propan-1-one hydrochloride;
[1-(3-Amino-2-{4-[2-(2,6-dichloro-phenoxy)-ethoxy]-benzyl}-propionyl)-pyrrolidin-3-yl]-carbamic acid tert-butyl ester;
2-Aminomethyl-3-{4-[2-(2,6-dichloro-phenoxy)-ethoxy]-phenyl}-1-(2,3-dihydro-indol-1-yl)-propan-1-one hydrochloride;
3-Amino-2-{4-[2-(2,4-difluoro-phenoxy)-ethoxy]-benzyl}-1-(6-trifluoromethoxy-3,4-dihydro-2H-quinolin-1-yl)-propan-1-one hydrochloride;
3-Amino-2-{4-[2-(2,4-difluoro-phenoxy)-ethoxy]-benzyl}-1-piperidin-1-yl-propan-1-one hydrochloride; and
3-Amino-2-{4-[2-(2,4-difluoro-phenoxy)-ethoxy]-benzyl}-1-(3,4-dihydro-2H-quinolin-1-yl)-propan-1-one hydrochloride.

7. The compound as claimed in claim 1 selected from 3-Amino-2-{4-[2-(2,4-difluoro-phenoxy)-ethoxy]-benzyl}-1-(2,3-dihydro-indol-1-yl)-propan-1-one hydrochloride;
3-Amino-2-{4-[2-(2,6-dichloro-phenoxy)-ethoxy]-benzyl}-1-(3-methoxymethyl-piperidin-1-yl)-propan-1-one hydrochloride;
3-Amino-2-{4-[2-(2,6-dichloro-phenoxy)-ethoxy]-benzyl}-1-piperidin-1-yl-propan-1-one hydrochloride;
1-(3-Amino-2-{4-[2-(2,6-dichloro-phenoxy)-ethoxy]-benzyl}-propionyl)-pyrrolidine-2-carboxylic acid methyl ester hydrochloride;
3-Amino-2-{4-[2-(2,6-dichloro-phenoxy)-ethoxy]-benzyl}-1-(3,4-dihydro-2H-quinolin-1-yl)-propan-1-one hydrochloride;
3-Amino-2-{4-[2-(2,6-dichloro-phenoxy)-ethoxy]-benzyl}-1-morpholin-4-yl-propan-1-one hydrochloride;
3-Amino-2-{4-[2-(2,6-dichloro-phenoxy)-ethoxy]-benzyl}-1-(6-methoxy-3,4-dihydro-2H-quinolin-1-yl)-propan-1-one hydrochloride;
3-Amino-2-{4-[2-(2,6-dichloro-phenoxy)-ethoxy]-benzyl}-1-pyrrolidin-1-yl-propan-1-one hydrochloride;
2-Aminomethyl-3-{6-[2-(2,6-dichloro-4-methyl-phenoxy)-ethoxy]-pyridin-3-yl}-1-(3-methoxymethyl-piperidin-1-yl)-propan-1-one hydrochloride;
2-Aminomethyl-3-{6-[2-(2,6-dichloro-phenoxy)-ethoxy]-pyridin-3-yl}-1-(3-methoxymethyl-piperidin-1-yl)-propan-1-one hydrochloride;
(R)-2-Aminomethyl-3-{4-[2-(2,6-dichloro-4-methyl-phenoxy)-ethoxy]-phenyl}-1-(2-methoxymethyl-pyrrolidin-1-yl)-propan-1-one hydrochloride; and
(S)-2-Aminomethyl-3-{4-[2-(2,6-dichloro-4-methyl-phenoxy)-ethoxy]-phenyl}-1-(2-methoxymethyl-pyrrolidin-1-yl)-propan-1-one hydrochloride.

8. (canceled)

9. (canceled)

10. (canceled)

11. (canceled)

12. (canceled)

13. A method of treating hypertension and associated disease conditions by administering a compound as claimed in claim 1 to a person in need thereof.

14. An intermediate of formula 5 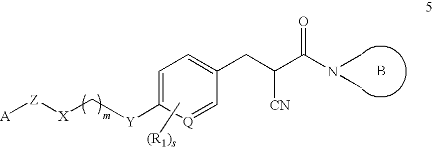
wherein A, Z, X, m, Y, R₁, Q and B are as defined in claim 1.

15. A pharmaceutical composition comprising an effective amount of a compound as claimed in claim 1.

16. A pharmaceutical composition comprising an effective amount of a compound as claimed in claim 6.

17. A pharmaceutical composition comprising an effective amount of a compound as claimed in claim 7.

18. A method of inhibiting rennin comprising administering an effective amount of a compound as claimed in claim 1 to a person in need thereof.
